(19) United States
(12) Patent Application Publication
(10) Pub. No.: US 2025/0232862 A1
(43) Pub. Date: Jul. 17, 2025

(54) METHODS AND SYSTEMS FOR MANAGING EXPOSURE AND RESPONSE PREVENTION (ERP) TREATMENT HEALTHCARE WORKFLOWS

(71) Applicants: Michael Kaufman, Rockford, IL (US); Julian Theuma, Marsaskala (MT)

(72) Inventors: Michael Kaufman, Rockford, IL (US); Julian Theuma, Marsaskala (MT)

(21) Appl. No.: 19/029,140

(22) Filed: Jan. 17, 2025

Related U.S. Application Data
(60) Provisional application No. 63/621,629, filed on Jan. 17, 2024.

(51) Int. Cl. G16H 20/40; G16H 10/60

(52) U.S. Cl. CPC G16H 20/40; G16H 10/60

(57) ABSTRACT

The subject matter described herein includes methods and systems for managing exposure and response prevention (ERP) treatment healthcare workflows of a patient. According to one computer-implemented method, healthcare data is received from a plurality of sources, where the healthcare data includes symptoms reported by the patient, treatment history, and clinician-provided instructions. The healthcare data is processed to generate a treatment plan that includes exposure assignments tailored to the patient, a sequence of ritual resistance tasks, and anxiety level monitoring parameters. The treatment plan is presented to the patient via a graphical user interface (GUI) configured to display progress metrics, upcoming tasks, and clinician feedback. Input is received from the patient indicating completion or modification of specific tasks within the treatment plan and the treatment plan is updated based on the received input to dynamically adjust subsequent assignments according to predefined treatment guidelines.

### CROSS-REFERENCE TO RELATED APPLICATIONS

This application claims the benefit of U.S. Provisional Patent Application No. 63/621,629 entitled “METHODS AND SYSTEMS FOR MANAGING EXPOSURE AND RESPONSE PREVENTION (ERP) TREATMENT HEALTHCARE WORKFLOWS” filed on Jan. 17, 2024; the entire contents of which are incorporated by reference herein.

### TECHNICAL FIELD

The present invention relates to healthcare, and more specifically, to managing exposure and response prevention (ERP) treatment healthcare workflows.

### BACKGROUND

Obsessive-compulsive disorder (OCD) affects 2.5 million adults or 1.2% of the U.S. population. At least 1 in 200—or 500,000-kids and teens have OCD. On average, people are diagnosed with OCD when they are 19 years old.

OCD is a long-lasting disorder in which a person experiences uncontrollable and recurring thoughts (obsessions), engages in repetitive behaviors (compulsions), or both, People with OCD may have obsessions, compulsions, or both that can cause significant distress or interfere with daily life.

Obsessions are repeated thoughts, urges, or mental images that are intrusive, unwanted, and make most people anxious. Common obsessions include fear of germs or contamination; fear of forgetting, losing, or misplacing something; fear of losing control over one's behavior; aggressive thoughts toward others or oneself; unwanted, forbidden, or taboo thoughts involving sex, religion, or harm; and desire to have things symmetrical or in perfect order.

Compulsions are repetitive behaviors a person feels the urge to do, often in response to an obsession. Common compulsions include excessive cleaning or handwashing; ordering or arranging items in a particular, precise way; repeatedly checking things, such as that the door is locked or the oven is off; compulsive counting; and praying or repeating words silently.

People with OCD might avoid situations that trigger their symptoms or use drugs or alcohol to cope. However, treatment is available to help people manage their symptoms and improve their quality of life.

Exposure and response prevention (ERP) therapy is one of the most effective forms of treatment for OCD. Under the guidance of mental health professionals, people who receive ERP therapy can gradually reduce their anxieties and stop the problematic cycle of OCD. ERP therapy is a behavioral therapy that gradually exposes people to situations designed to provoke a person's obsessions in a safe environment. ERP therapy may also be used to treat other mental health conditions such as Generalized Anxiety Disorder (GAD) and Post-traumatic Stress Disorder (PTSD).

Currently, the treatment of, and records for, patients with OCD seeking treatment through ERP therapy in a clinical or individualized setting has remained largely primitive. Kitchen timers are used to track elapsed time and records are kept on paper. This leads to a bulky load of materials for patients to haul around and makes engaging in treatment at public places inconvenient and far from inconspicuous. It also leaves clinicians with two unsavory options: to re-record all their client's progress digitally or to let the documentation disappear.

As may be appreciated from these examples, current methods and systems for managing ERP therapy healthcare workflows are prone to errors, inefficiencies, sub-optimal outcomes, and other drawbacks.

Accordingly, a need exists for improved systems and methods for managing ERP therapy healthcare workflows that address these deficiencies.

### SUMMARY

The subject matter described herein includes methods and systems for managing exposure and response prevention (ERP) treatment healthcare workflows of a patient. According to one computer-implemented method, healthcare data is received from a plurality of sources, where the healthcare data includes symptoms reported by the patient, treatment history, and clinician-provided instructions. The healthcare data is processed to generate a treatment plan that includes exposure assignments tailored to the patient, a sequence of ritual resistance tasks, and anxiety level monitoring parameters. The treatment plan is presented to the patient via a graphical user interface (GUI) configured to display progress metrics, upcoming tasks, and clinician feedback. Input is received from the patient indicating completion or modification of specific tasks within the treatment plan and the treatment plan is updated based on the received input to dynamically adjust subsequent assignments according to predefined treatment guidelines.

A system for managing exposure and response prevention (ERP) treatment healthcare workflows includes a computing device communicatively coupled to a database and is to receive healthcare data from a plurality of sources, including data provided by the patient and clinician records. The system processes the healthcare data to generate a treatment plan that includes a sequence of exposure assignments, ritual resistance tracking, and anxiety level monitoring parameters. The system presents the treatment plan via a graphical user interface (GUI) configured for interaction by the patient, clinicians, and administrators. The system then receives user input indicating task completion or modification and updates the treatment plan dynamically based on predefined rules and the received input, and the updated plan is stored in the database for subsequent access and modification.

### BRIEF DESCRIPTION OF THE DRAWINGS

FIG. 1 is a flow diagram illustrating exemplary steps for managing healthcare workflows according to an embodiment of the subject matter described herein.

FIG. 2 is a system diagram illustrating exemplary functional components for managing healthcare workflows according to an embodiment of the subject matter described herein.

FIG. 3 is a diagram illustrating an exemplary data flow for managing healthcare workflows according to an embodiment of the subject matter described herein.

FIG. 4 is a diagram illustrating an exemplary Software-as-a-Service (SaaS) platform suitable for managing healthcare workflows according to an embodiment of the subject matter described herein.

FIGS. 5A, 5B, and 5C are diagrams illustrating exemplary user information intake forms for managing healthcare workflows according to an embodiment of the subject matter described herein.

FIG. 6 is a diagram illustrating an exemplary home screen interface for managing healthcare workflows according to an embodiment of the subject matter described herein.

FIG. 7 is a diagram illustrating an exemplary user interface for displaying medication information as part of managing healthcare workflows according to an embodiment of the subject matter described herein.

FIG. 8 is a diagram illustrating an exemplary user interface for displaying assignment information as part of managing healthcare workflows according to an embodiment of the subject matter described herein.

FIGS. 9A and 9B are diagrams illustrating exemplary user interfaces for presenting a list of assigned exposures and displaying instructions for a selected exposure, respectively, according to an embodiment of the subject matter described herein.

FIGS. 9C, 9D, 9E, and 9F are diagrams illustrating an exemplary user interface sequence for tracking a user anxiety level before and after completing an assigned exposure task as part of managing healthcare workflows according to an embodiment of the subject matter described herein.

FIGS. 10A, 10B, and 10C are diagrams illustrating exemplary user interfaces for creating, adding, and sharing journal entries as part of managing healthcare workflows according to an embodiment of the subject matter described herein.

FIGS. 11A and 11B are diagrams illustrating exemplary user interfaces for selecting and recording progress with ritual resistance as part of managing healthcare workflows according to an embodiment of the subject matter described herein.

FIGS. 11C, 11D, and 11E are diagrams illustrating exemplary user interfaces for recording a user submission, resistance, and undoing the saving of a record as part of managing healthcare workflows according to an embodiment of the subject matter described herein.

FIGS. 12A, and 12B are diagrams illustrating an exemplary user interface for sending and receiving direct messages between users and care team members according to an embodiment of the subject matter described herein.

FIG. 12C is a diagram illustrating an exemplary user interface for emergency services according to an embodiment of the subject matter described herein.

FIG. 13A is a diagram illustrating an exemplary home screen user interface for clinicians according to an embodiment of the subject matter described herein.

FIG. 13B is a diagram illustrating an exemplary appointments user interface for clinicians according to an embodiment of the subject matter described herein.

FIG. 13C is a diagram illustrating an exemplary emergency widget user interface for clinicians according to an embodiment of the subject matter described herein.

FIGS. 14A, 14B, and 14C are diagrams illustrating exemplary user interfaces for allowing clinicians to view currently assigned exposures, assign new exposures, and archive completed exposures according to embodiments of the subject matter described herein.

FIG. 14D is a diagram illustrating an exemplary user interface allowing clinicians to view client progress according to an embodiment of the subject matter described herein.

FIGS. 15A, and 15B are diagrams illustrating exemplary user interfaces for allowing clinicians to assign new ritual resistance categories and archive completed assignments according to embodiments of the subject matter described herein.

FIG. 15C is a diagram illustrating an exemplary user interface for allowing clinicians to view client progress on ritual resistances according to an embodiment of the subject matter described herein.

FIG. 15D is a diagram illustrating an exemplary feedback process according to an embodiment of the subject matter described herein.

FIGS. 16A and 16B are diagrams illustrating exemplary user interfaces for allowing clinicians to view any journals shared by a client or patient according to embodiments of the subject matter described herein.

FIGS. 17A and 17B are diagrams illustrating exemplary user interfaces for allowing clinicians to view upcoming and completed appointments, view detailed appointment information, and schedule or cancel appointments according to embodiments of the subject matter described herein.

FIGS. 18A and 18B are diagrams illustrating exemplary user interfaces for accessing a patient's medications and medical information according to embodiments of the subject matter described herein.

FIGS. 19A and 19B are diagrams illustrating exemplary user interfaces for uploading, creating, or editing forms and assigning forms to patients according to embodiments of the subject matter described herein.

FIG. 20 is a diagram illustrating an exemplary user interface for adding or removing a user account as a member of a client's care team according to embodiments of the subject matter described herein.

FIG. 21 is a diagram illustrating an exemplary user interface for logging client billing information according to embodiments of the subject matter described herein.

FIG. 22 is a diagram illustrating an exemplary user interface for accessing emergency resources according to embodiments of the subject matter described herein.

FIGS. 23A and 23B are diagrams illustrating exemplary user interfaces for managing administrator functions according to embodiments of the subject matter described herein.

FIGS. 24A, 24B, 24C, 24D, and 24E are diagrams illustrating exemplary user interfaces for viewing a clinician's profile, adding appointments and events to a schedule, updating records, and viewing a clinician's specialties according to embodiments of the subject matter described herein.

FIGS. 25A, 25B, and 25C are diagrams illustrating exemplary user interfaces for viewing, adding, and editing hospital information according to embodiments of the subject matter described herein.

FIG. 26 is a diagram illustrating an exemplary user interface for accessing emergency resources according to embodiments of the subject matter described herein.

### DETAILED DESCRIPTION

The following description and figures are illustrative and are not to be construed as limiting. Numerous specific details are described to provide a thorough understanding of the disclosure. In certain instances, however, well-known or conventional details are not described in order to avoid obscuring the description. References to “one embodiment” or “an embodiment” in the present disclosure may be (but are not necessarily) references to the same embodiment, and such references mean at least one of the embodiments.

Reference in this specification to “one embodiment” or “an embodiment” means that a particular feature, structure, or characteristic described in connection with the embodiment is included in at least one embodiment of the disclosure. Multiple appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others. Similarly, various requirements are described which may be requirements for some embodiments but not for other embodiments.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that same thing can be said in more than one way.

Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification, including examples of any terms discussed herein, is illustrative only, and is not intended to further limit the scope and meaning of the disclosure or of any exemplified term. Likewise, the disclosure is not limited to various embodiments given in this specification.

Without intent to limit the scope of the disclosure, examples of instruments, apparatus, methods and their related results according to the embodiments of the present disclosure are given below. Note that titles or subtitles may be used in the examples for convenience of a reader, which in no way should limit the scope of the disclosure. Unless otherwise defined, all technical and scientific terms used herein have the same meaning as commonly understood by one of ordinary skill in the art to which this disclosure pertains. In the case of conflict, the present document, including definitions, will control.

In contrast to conventional methods and systems for managing the treatment of and records for patients with OCD and other mental health conditions which rely on primitive kitchen timers and paper records, the present disclosure includes a suite of software tools necessary for measuring all exposures and activities (including a timer, a scale measuring current distress levels, a compulsion/ritual prevention record, an individualized hierarchy of fears, and journal submissions that can either be kept private to the client or shared with their clinician), and archiving all previous work. Data for the system is routed through and saved by HIPAA-compliant servers.

The subject matter described herein includes a combination of hardware and software components to implement a system for managing healthcare workflows, particularly exposure and response prevention (ERP) therapy for mental health conditions. Unlike conventional systems that rely on generic timers or manual record-keeping, this system provides a tangible technological solution by integrating real-time data collection, machine learning-based analysis, and dynamic user interfaces. These elements collectively ensure that the system is rooted in the practical application of computing technology to address specific challenges in healthcare delivery.

A feature of the system is its use of real-time patient data to dynamically adjust treatment plans. This involves the collection of data through patient interactions with a graphical user interface (GUI), such as inputting anxiety levels or tracking ritual resistance behaviors. The system processes this data using predefined algorithms or machine learning models to generate tailored exposure assignments or modify therapeutic tasks, which enhances the precision and effectiveness of healthcare workflows.

The system's integration of hardware components, such as HIPAA-compliant servers, and software modules, including cloud-based data storage and analysis tools, provides secure and efficient handling of sensitive healthcare data. By routing data through the multi-layered architecture, which spans infrastructure, platform, and software-as-a-service (SaaS) models, further highlights the invention's technical implementation, the system provides scalability, data integrity, and compliance with regulatory standards.

Additionally, the system enhances the usability and accessibility of healthcare workflows by providing role-specific GUIs for patients, clinicians, and administrators. Each interface is designed to facilitate specific tasks, such as monitoring progress, assigning new exposures, or managing billing. These GUIs are integrated with backend systems that analyze data and trigger actionable insights. For example, the clinician's dashboard uses progress charts and patient logs to identify trends and inform treatment decisions. This functionality is a technical improvement to the way healthcare data is analyzed and utilized.

The system also uses new methods for tracking and visualizing patient progress. Features such as the anxiety tracking module, ritual resistance tally, and dynamic progress indicators enable granular and real-time monitoring of therapeutic outcomes. These features represent concrete advancements in the way data is captured, processed, and presented to end users. The use of timers, sliders, and numerical scales to measure anxiety levels and task completion further emphasizes the technical solution embodied by the system.

By addressing specific, real-world challenges in mental healthcare, the system demonstrates a clear practical application. Its technical implementation of machine learning, real-time data processing, and role-specific interfaces transforms traditional, paper-based processes into a streamlined, data-driven solution.

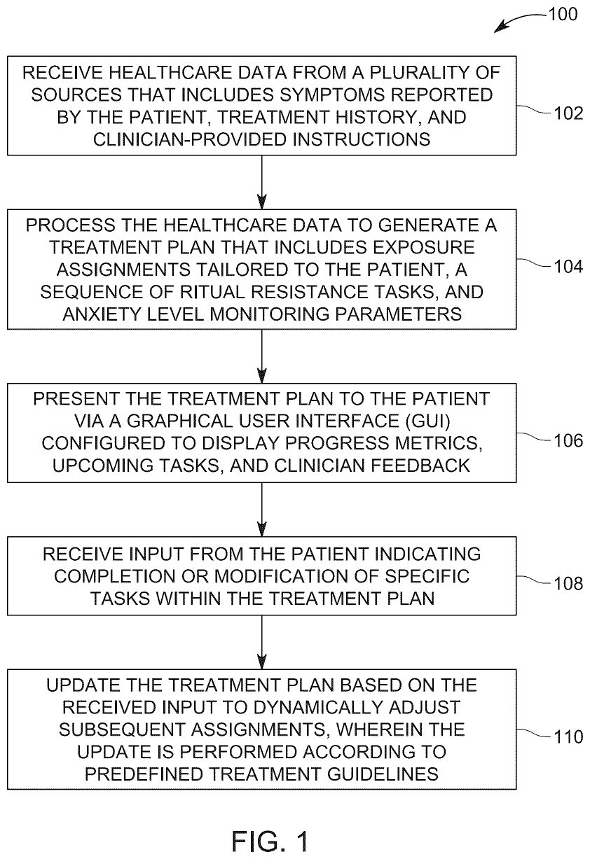

FIG. 1 is a flow diagram illustrating exemplary steps of a computer-implemented method for managing exposure and response prevention (ERP) treatment healthcare workflows of a patient according to an embodiment of the subject matter described herein.

At step 102, healthcare data is received from a plurality of sources, where the healthcare data includes symptoms reported by the patient, treatment history, and clinician-provided instructions. The healthcare data may include biographical information, treatment goals, and digital journaling entries submitted by the patient and the healthcare data may be supplemented by external sources, including medication records, clinician notes, and appointment schedules.

Obtaining the healthcare data may include creating, editing, and/or presenting a user intake form to a patient or client. Intake forms may be used to screen for applicable medical conditions. Screening for all applicable medical conditions includes, but is not limited to, screening for: Obsessive Compulsive Disorder, Generalized Anxiety Disorder, and Post-traumatic Stress Disorder. Specific examples of medical screening or intake forms include, but are not limited to: DASS Adult, DASS Youth, Y-BOCS (Child), Y-BOCS (Adult), Dimensional Obsessive-Compulsive Scale, Patient Health Questionnaire-2 (PHQ-2), Patient Health Questionnaire-9 (PHQ-9), Generalized Anxiety Disorder Scale-7 (GAD-2), Generalized Anxiety Disorder Scale-7 (GAD-7), Obsessive-Compulsive Inventory-revised (OCI-R), and World Health Organization Well-Being Index (WHO-5).

Additionally, as will be described in greater detail below, healthcare data may also include information received from electronic data sources, such as medication, hospital, or clinic databases. This information may be received as user input or from an existing database of information. For example, the software suite includes program views for three different participants: patients, clinicians, and administrators. Patient input may include biographical information such as name, address, phone number, and other contact information. Patient input may also include appointments (e.g., synchronized calendar), treatment goals, journal entries, and progress reports. Medical information databases may provide information such as medication information and contact information for medical referrals. Administration information may include patient names, calendar information, direct messages, billing, and clinic or doctor accreditations. Clinician or doctor information may include exposures, ban books, journals, appointments, patient medical information, forms, and billing. Hospital information may include how much has been paid, insurance or co-insurance details, deductible amount, and claim number. Other back end or cloud-based data may include software updates and mobile applications.

At step 104, the healthcare data is processed to generate a treatment plan that includes exposure assignments tailored to the patient, a sequence of ritual resistance tasks, and anxiety level monitoring parameters. Processing the healthcare data may include correlating patient-reported anxiety levels with historical treatment data to determine an optimal exposure intensity.

The analysis of the healthcare data may be, for example, based on the patient's medical history and clinician input, one or more exposure assignments may be determined. The exposure assignments are designed to help the patient confront and reduce the fear and anxiety triggered by specific situations, objects, or thoughts. One example includes a fear of dogs. Exposure assignments may include thinking about petting a dog, looking at a picture of a dog and thinking about petting it, and finding a dog and petting it.

Exposure assignments may also include one or more detailed steps. For example, the assignment to pet a dog may be broken down into four instructions: visit a local park, find a small dog, approach the dog, and pet the dog for five seconds. In one embodiment, for the purposes of treatment, all phases must be completed for a successful outcome. This may or may not be necessary to indicate. In one example, the patient may not mark the completion of just one task because all steps must be taken for a successful trial. The purpose of using added or intermediary steps is to allow for clear and detailed instructions to be created by the patient and/or clinician, and for clarity on behalf of the patient during the completion of exposures. In other embodiments, the user can mark intermediary steps as complete, or the user can more thoroughly engage with intermediary steps in other ways.

The use of intermediary steps may allow the system to determine whether the patient successfully completed all the instructions or failed on a specific instruction. This may help a doctor or other member of the care team adjust a treatment plan for the patient. For example, if the patient repeatedly fails to accomplish the assigned task of petting a dog, it may be important to distinguish between the patient petting the dog, but for less than five seconds and never attempting to find a dog at all. In the former scenario, the patient has accomplished virtually all steps of the assigned task while in the latter scenario, the patient has not accomplished any of the steps of the task. This may be tracked as progress, even though in both scenarios the patient failed to successfully complete the entire task.

In another embodiment, patient input may be used to determine or adjust a treatment plan. For example, the patient may input their self-reported anxiety level (e.g., translated into a numerical scale of severity) when performing a task. The patient may then perform the task for an amount of time that is measured and/or determined using a digital timer. Afterwards, the patient may again input their anxiety level. If the patient reports low levels of anxiety before and after performing the task for a period of time, the system may determine that an increase in the amount of time is warranted until the patient's anxiety level reaches a threshold level, after which the time period may be stabilized or reduced.

It may be appreciated that the act of recording the levels of anxiety an individual experiences before and after an exposure is completed may be to measure whether an adequate amount of time has been spent on an exposure for the anxiety to peak and wane, as well as to determine when an obsessive thought, fear, or compulsion has been successfully quashed. In some embodiments, for the exposure to be considered a success, the ending anxiety level recorded must be at least 50% less than the initial level of anxiety experienced. Once the starting anxiety is low after consistent trials of an exposure over a course of days or weeks, the patient may move on to more challenging exposures.

In another embodiment, the system may obtain journal entries from the patient. The system may analyze this information in combination with the patient's schedule and medication information, as well as other input, to determine or adjust a treatment plan. For example, the system may know that the patient began taking a medication at a certain day or time of day and that the patient also reported in their journal that their symptoms improved (or deteriorated) shortly thereafter. The system may determine that the change in symptoms reported by the patient in their journal are correlated with, or caused by, the medication and may adjust accordingly (e.g., reduced the dosage or change the medication).

At step 106, the treatment plan is presented to the patient via a graphical user interface (GUI) configured to display progress metrics, upcoming tasks, and clinician feedback. The GUI may include separate views optimized for patients, clinicians, and administrators, each tailored to display role-specific information. The GUI may also include a visual progress tracker that graphically represents the patient's completion of assigned tasks over time.

Treatment plan updates may be stored in a database compliant with healthcare privacy standards, enabling remote access for authorized users

The user interface provides for viewing and managing the healthcare data. In one embodiment, providing the interface for viewing and managing the healthcare data includes displaying a GUI to a user. Details and example screenshots illustrating a GUI for managing healthcare workflows will be described in greater detail below.

The interface may be presented differently depending on the client device and/or the user. For example, a patient portal mobile interface may be presented to a patient when they log into the system on their mobile device using their account information. A clinician portal desktop, or web-based, interface may be presented to a clinician when they log into their account on a desktop or laptop computer. It is appreciated that the clinician portal may include features or elements that are not included in the patient portal, and vice versa. For example, the patient portal may include a journaling feature for writing journal entries for only that patient while the clinician portal may include a journal review feature for reading journal entries for multiple patients under their care. This same principle applies to other types of devices and/or user accounts, such as administrators.

In one embodiment, automated alerts may also be provided to a clinician when a patient fails to complete an assigned task within a designated timeframe.

At step 108, input is received from the patient indicating completion or modification of specific tasks within the treatment plan and the treatment plan. The received input may include a user-selected indication of ritual resistance or submission during a specific exposure assignment.

The instructions for performing an action received from the patient may be received using, for example, the mobile app patient interface may include options for recording instances of either resistance or submission to a particular anxiety trigger (e.g., dogs). The input received from the user includes clicking a button indicating whether the user resisted or submitted. The action taken in response may be to record the time and location of the indication as well as a total tally of indications over a period of time. Actions may also include providing this data to other members of the care team or storing it locally and/or remotely in one or more data storage locations.

As used herein, when a user “submits”, they are engaging in either avoidance, safety, or self-comforting behaviors that are detrimental to their treatment. When a user “resists”, they are refraining from giving in to their compulsions or self-comforting behaviors. When a user “undo” or “undoes”, the user is engaging in a self-comforting behavior that undermines the treatment progress made through a previous “resist” tally. This allows a provider to understand how their client is progressing in their day-to-day life outside of specific times when the client is engaging in ERP treatment (i.e. when they are at work, school, or a social function).

In other embodiments, such as one where the user is an external client such as a provider and the interface provided is a “Provider Portal” or “Provider Dashboard”, the user may check eligibility, receive documents uploaded by another user, provide a communication log, generate a status report, or check compliance.

At step 110, the treatment plan is updated based on the received input to dynamically adjust subsequent assignments according to predefined treatment guidelines. The updated treatment plan may include new exposure tasks derived from a hierarchy of fears established during an initial patient assessment.

The action performed may be based on the instructions, the healthcare data, and the analysis. For example, the action may include: generating one or more documents, updating a task manager, updating a communication log, generating a report, generating client invoicing, checking eligibility, receiving documents uploaded by another user, providing a communication log, generating a status report, and checking compliance.

It is appreciated that some actions may include viewing or managing data internal to the system, such as calendar, tasks, and reports, while other actions may include sending or receiving data from external sources. As previously mentioned, receiving data from external sources may involve various data aggregation, formatting, translation, or normalization procedures. It may also be appreciated that that this may potentially include importing data from or exporting data to other EHR systems, such as Epic.

Actions that involve sending data to external sources may often include an intermediary step of generating the data to be sent. In some embodiments, data saved by patients is available either by manual push request or automatic data sharing (cloud or otherwise) in order to be received by the provider.

According to one aspect, a trained machine learning model is used to analyze the healthcare data. The system employs a model stored in memory and executed by the hardware processor. Using machine learning-particularly the detection of patient-specific anxiety triggers and the prediction of changes in anxiety levels—is more than merely collecting and processing patient data.

Additionally, by specifically describing “exposure assignments,” “ritual resistance tasks,” and “anxiety level monitoring,” the claims are directed toward a particular medical practice rather than broadly reciting any healthcare workflow. These details also underscore how the invention provides a tailored solution for managing ERP therapy sessions.

According to one aspect, the presence of a hardware processor, memory, and a database (along with explicit statements on how these components interact), storing the machine learning model in memory, and specifying that it operates on real-time and historical data demonstrates how the system functions as a specialized tool rather than performing mental steps or abstract calculations.

Additionally, the system's dynamic, real-time adaptation capabilities allow the model to refine its predictions and adjust treatment scheduling or intensity upon receiving new user input. Thus, the subject matter disclosed herein is a continuously evolving platform that responds to current patient conditions.

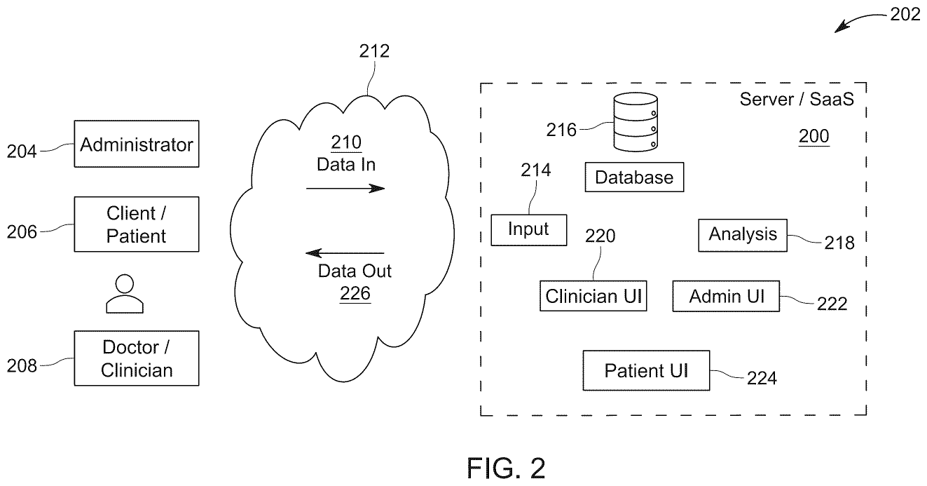

FIG. 2 is a system diagram illustrating exemplary functional components for managing healthcare workflows according to an embodiment of the subject matter described herein. The system 200 includes functions executed in software by a computing device connected to a network. The computing device can include one or more servers that can be located locally or remotely from users.

The system disclosed herein may be implemented as a client/server type architecture but may also be implemented using other architectures, such as cloud computing, software as a service model (SaaS), a mainframe/terminal model, a stand-alone computer model, a plurality of non-transitory lines of code on a computer readable medium that can be loaded onto a computer system, a plurality of non-transitory lines of code downloadable to a computer, and the like. In one embodiment, the system 200 may be hosted in the cloud as Software as a Service (SaaS).

The system may be implemented as one or more computing devices that connect to, communicate with and/or exchange data over a link that interact with each other. Each computing device may be a processing unit-based device with sufficient processing power, memory/storage and connectivity/communications capabilities to connect to and interact with the system. For example, each computing device may be an Apple iPhone or iPad product, a Blackberry or Nokia product, a mobile product that executes the Android operating system, a personal computer, a tablet computer, a laptop computer and the like and the system is not limited to operate with any particular computing device. The link may be any wired or wireless communications link that allows the one or more computing devices and the system to communicate with each other. In one example, the link may be a combination of wireless digital data networks that connect to the computing devices and the Internet. The system may be implemented as one or more server computers (all located at one geographic location or in disparate locations) that execute a plurality of lines of non-transitory computer code to implement the functions and operations of the system as described herein. Alternatively, the system may be implemented as a hardware unit in which the functions and operations of the back-end system are programmed into a hardware system. In one implementation, the one or more server computers may use Intel® processors, run the Linux operating system, and execute Java, Ruby, Regular Expression, Flex 4.0, SQL etc.

In some embodiments, each computing device may further comprise a display and a browser application so that the display can display information generated by the system. The browser application may be a plurality of non-transitory lines of computer code executed by a processing unit of the computing device. Each computing device may also have the usual components of a computing device such as one or more processing units, memory, permanent storage, wireless/wired communication circuitry, an operating system, etc.

The system may further comprise a server (that may be software based or hardware based) that allows each computing device to connect to and interact with the system such as sending information and receiving information from the computing devices that is executed by one or more processing units. The system may further comprise software—or hardware-based modules and database(s) for processing and storing content associated with the system, metadata generated by the system for each piece of content, user preferences, and the like.

In one embodiment, the system includes one or more processors, server, clients, data storage devices, and non-transitory computer readable instructions that, when executed by a processor, cause a device to perform one or more functions. It is appreciated that the functions described herein may be performed by a single device or may be distributed across multiple devices.

When a user interacts with the system, the user may use a frontend client application. The client application may include a graphical user interface that allows the user to select one or more digital files. The client application may communicate with a backend cloud component using an application programming interface (API) comprising a set of definitions and protocols for building and integrating application software. As used herein, an API is a connection between computers or between computer programs that is a type of software interface, offering a service to other pieces of software. A document or standard that describes how to build or use such a connection or interface is called an API specification. A computer system that meets this standard is said to implement or expose an API. The term API may refer either to the specification or to the implementation.

Software-as-a-service (SaaS) is a software licensing and delivery model in which software is licensed on a subscription basis and is centrally hosted. SaaS is typically accessed by users using a thin client, e.g., via a web browser. SaaS is considered part of the nomenclature of cloud computing.

Many SaaS solutions are based on a multitenant architecture. With this model, a single version of the application, with a single configuration (hardware, network, operating system), is used for all customers (“tenants”). To support scalability, the application is installed on multiple machines (called horizontal scaling). The term “software multitenancy” refers to a software architecture in which a single instance of software runs on a server and serves multiple tenants. Systems designed in such manner are often called shared (in contrast to dedicated or isolated). A tenant is a group of users who share a common access with specific privileges to the software instance. With a multitenant architecture, a software application is designed to provide every tenant a dedicated share of the instance-including its data, configuration, user management, tenant individual functionality and non-functional properties.

The backend cloud component described herein may also be referred to as a SaaS component. One or more tenants may communicate with the SaaS component via a communications network, such as the Internet. The SaaS component may be logically divided into one or more layers, each layer providing separate functionality and being capable of communicating with one or more other layers.

Cloud storage may store or manage information using a public or private cloud. Cloud storage is a model of computer data storage in which the digital data is stored in logical pools. The physical storage spans multiple servers (sometimes in multiple locations), and the physical environment is typically owned and managed by a hosting company. Cloud storage providers are responsible for keeping the data available and accessible, and the physical environment protected and running. People and/or organizations buy or lease storage capacity from the providers to store user, organization, or application data. Cloud storage services may be accessed through a co-located cloud computing service, a web service API, or by applications that utilize the API.

In an exemplary SaaS model, the functionality disclosed herein can be logically divided into layers. Layers may be ordered from least to greatest abstraction of underlying physical resources. Layers may also be divided into groups based on common features for simplicity when referring or billing functions associated with each group.

Infrastructure may include a storage function, a networking function, a server function, and a virtualization function. Infrastructure functions may be bundled together and provided to one or more tenants as a service, referred to as Infrastructure-as-a-Service (IaaS). IaaS is made up of a collection of physical and virtualized resources that provide consumers with the basic building blocks needed to run applications and workloads in the cloud.

The storage function provides storage of data without requiring the user or tenant to be aware of how this data storage is managed. Three types of cloud storage that may be provided by the storage function are: block storage, file storage, and object storage. Object storage is the most common mode of storage in the cloud because it is highly distributed (and thus resilient), data can be accessed easily over HTTP, and performance scales linearly as the storage grows.

The networking function in the cloud is a form of software defined networking in which traditional networking hardware, such as routers and switches, are made available programmatically, typically through APIs.

The server function refers to various physical hardware resources associated with executing computer-readable code that is not otherwise part of the virtualized network resources in the networking function or the storage function. IaaS providers manage large data centers, typically around the world, that contain the servers powering the various layers of abstraction on top of them and that are made available to end users. In most IaaS models, end users do not interact directly with the physical infrastructure (e.g., memory, motherboard, CPU), but it is provided as a service to them.

The virtualization function provides virtualization of underlying resources via one or more virtual machines (VMs). Virtualization relies on software to simulate hardware functionality and create a virtual computer system. A virtual computer system is known as a “virtual machine” (VM): a tightly isolated software container with an operating system and application inside. Each self-contained VM is completely independent. Putting multiple VMs on a single computer enables several operating systems and applications to run on just one physical server, or “host.” A thin layer of software called a “hypervisor” decouples the virtual machines from the host and dynamically allocates computing resources to each virtual machine as needed. Providers manage hypervisors and end users can then programmatically provision virtual “instances” with desired amounts of compute and memory (and sometimes storage). Most providers offer both CPUs and GPUs for different types of workloads.

The platform may include an operating system, middleware, and runtime. The infrastructure functions and the platform functions may be bundled together and provided to one or more tenants as a service, referred to as Platform-as-a-Service (PaaS). In the Platform-as-a-Service (PaaS) model, developers rent everything needed to build an application, relying on a cloud provider for development tools, infrastructure, and operating systems.

The operating system function refers to a PaaS vendor providing and maintaining the operating system that developers use, and the application runs on. For example, Windows and Linux operating systems may be installed in virtual machines and Windows or Linux applications may be run within the operating system.

The middleware is software that sits in between user-facing applications and the machine's operating system. For example, middleware may allow software to access input from the keyboard and mouse. Middleware is necessary for running an application but end users don't interact with it. Relatedly, the middleware function may also include tools that are necessary for software development, such as a source code editor, a debugger, and a compiler. These tools may be offered together as a framework.

The runtime is software code that implements portions of a programming language's execution model. A runtime system or runtime environment implements portions of an execution model. Most programming languages have some form of runtime system that provides an environment in which programs run. This environment may address issues including the management of application memory, how the program accesses variables, mechanisms for passing parameters between procedures, interfacing with the operating system, and otherwise. The compiler makes assumptions depending on the specific runtime system to generate correct code. Typically, the runtime system will have some responsibility for setting up and managing the stack and heap, and may include features such as garbage collection, threads or other dynamic features built into the language.

The software includes applications and data functions. The infrastructure, platform, and software may be bundled together and provided to one or more tenants as a service, referred to as Software-as-a-Service (SaaS). Applications and data refer to the application and associated data created and managed by the user. For example, an application programmed by the user for providing functionality disclosed herein and may be exposed to the end user via a front-end interface such as a web browser or dedicated front-end client application. Neither the front-end user nor the back-end developer is required to manage or maintain services provided by the platform and the infrastructure. This contrasts with on-site hosting of the same functionality.

The system 200 may receive data from one or more external data sources 202. These external data sources 202 may include, for example, administrator 204, client or patient 206, doctor or clinician 208.

Data 210 may be communicated to server 200 via one or more networks 212, such as the Internet.

Data 210 may be received by an input module 214 and stored in a database 216. Database 216 may be accessed by one or more modules of the system 200. For example, an administrative user interface (UI) module may access database 216 for administrative uses such as error logs, software updates, and adding or removing users.

Clinician UI module 220 may access database 216 for displaying healthcare data to a user via a GUI such as a dashboard. For example, the clinician UI module 220 may generate and display a provider/external client dashboard. Via the provider/external client dashboard, a provider or external client may perform various actions on the data in database 216, also referred to as applications.

In addition to the unified portal for viewing data stored in database 216 that is part of a healthcare workflow, the system 200 may also include an applications module for performing an action based on received instructions and the data stored in the database 216.

In addition to the provider or external client application mentioned above, a user dashboard may allow other users to perform various actions.

An analysis module 228 is configured to analyze the data in database 216. Analysis module 228 may be configured to analyze the healthcare data and/or generate new or additional data which may also be stored in database 216.

Machine learning (ML) is the use of computer algorithms that can improve automatically through experience and using data. Machine learning algorithms build a model based on sample data, also known as training data, to make predictions or decisions without being explicitly programmed to do so. Machine learning algorithms are used where it is unfeasible to develop conventional algorithms to perform the needed tasks.

In certain embodiments, instead of, or in addition to, performing the functions described herein manually, the system may perform some or all the functions using machine learning or artificial intelligence. Thus, in certain embodiments, machine learning-enabled software relies on unsupervised and/or supervised learning processes to perform the functions described herein in place of a human user.

Machine learning may include identifying one or more data sources and extracting data from the identified data sources. Instead of or in addition to transforming the data into a rigid, structured format, in which certain metadata or other information associated with the data and/or the data sources may be lost, incorrect transformations may be made, or the like, machine learning-based software may load the data in an unstructured format and automatically determine relationships between the data. Machine learning-based software may identify relationships between data in an unstructured format, assemble the data into a structured format, evaluate the correctness of the identified relationships and assembled data, and/or provide machine learning functions to a user based on the extracted and loaded data, and/or evaluate the predictive performance of the machine learning functions (e.g., “learn” from the data).

In certain embodiments, machine learning-based software assembles data into an organized format using one or more unsupervised learning techniques. Unsupervised learning techniques can identify relationships between data elements in an unstructured format.

In certain embodiments, machine learning-based software can use the organized data derived from the unsupervised learning techniques in supervised learning methods to respond to analysis requests and to provide machine learning results, such as a classification, a confidence metric, an inferred function, a regression function, an answer, a prediction, a recognized pattern, a rule, a recommendation, or other results. Supervised machine learning, as used herein, comprises one or more modules, computer executable program code, logic hardware, and/or other entities configured to learn from or train on input data, and to apply the learning or training to provide results or analysis for subsequent data.

Machine learning-based software may include a model generator, a training data module, a model processor, a model memory, and a communication device. Machine learning-based software may be configured to create prediction models based on the training data. In some embodiments, machine learning-based software may generate decision trees. For example, machine learning-based software may generate nodes, splits, and branches in a decision tree. Machine learning-based software may also calculate coefficients and hyper parameters of a decision tree based on the training data set. In other embodiments, machine learning-based software may use Bayesian algorithms or clustering algorithms to generate predicting models. In yet other embodiments, machine learning-based software may use association rule mining, artificial neural networks, and/or deep learning algorithms to develop models. In some embodiments, to improve the efficiency of the model generation, machine learning-based software may utilize hardware optimized for machine learning functions, such as an FPGA.

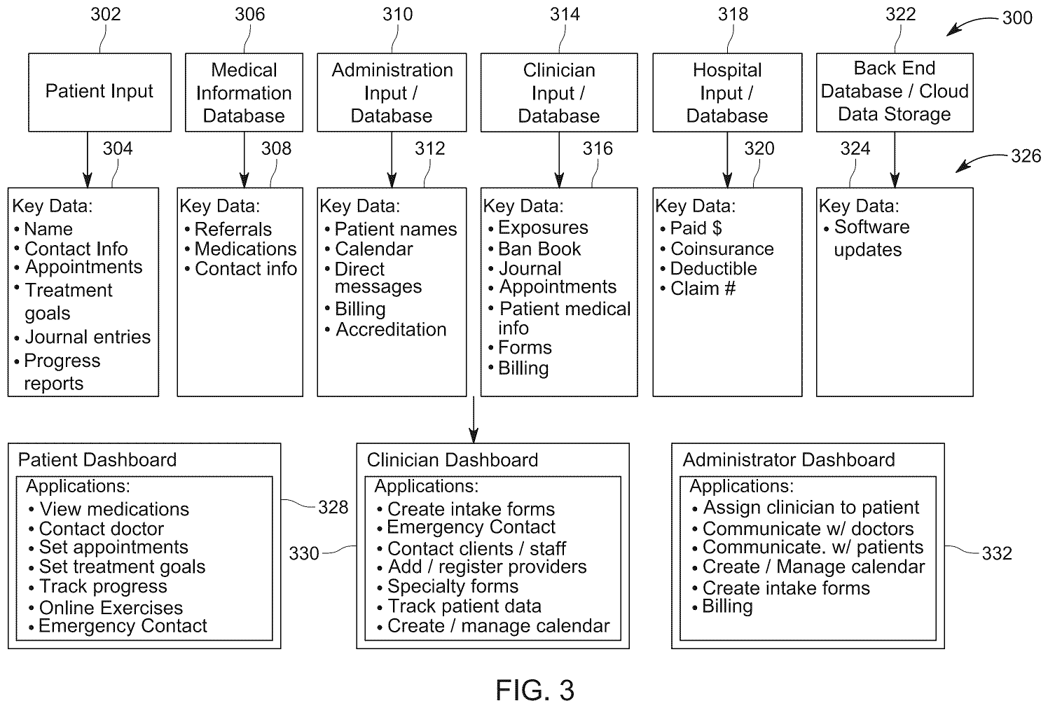

FIG. 3 is a diagram illustrating an exemplary data flow for managing healthcare workflows according to an embodiment of the subject matter described herein.

Exemplary data sources 300 include: patient input 302, medical information 306, administration information or input 310, clinician information or input 314, hospital information or input 318, and back-end or cloud storage data 322.

Patient input 302 may include key data 304, such as biographical information such as name, address, phone number, and other contact information. Patient input may also include appointments (e.g., synchronized calendar), treatment goals, journal entries, and progress reports.

Medical information 306 may include key data 308, such as medication information and contact information for medical referrals.

Administration information 310 may include key data 312, such as patient names, calendar information, direct messages, billing, and clinic or doctor accreditations.

Clinician or doctor information 314 may include key data 316, such as exposures, ban books, journals, appointments, patient medical information, forms, and billing. Examples of medical screening or intake forms include, but are not limited to: DASS Adult, DASS Youth, Y-BOCS (Child), Y-BOCS (Adult), Dimensional Obsessive-Compulsive Scale, Patient Health Questionnaire-2 (PHQ-2), Patient Health Questionnaire-9 (PHQ-9), Generalized Anxiety Disorder Scale-7 (GAD-2), Generalized Anxiety Disorder Scale-7 (GAD-7), Obsessive-Compulsive Inventory-revised (OCI-R), and World Health Organization Well-Being Index (WHO-5).

Hospital information 318 may include key data 320, such as how much has been paid, insurance or co-insurance details, deductible amount, and claim number. Other back end or cloud-based data may include software updates and mobile applications.

Back-end or cloud storage data 322 may include key data 324, such as software updates.

As mentioned above, a user interface may be presented differently depending on the client device and/or the user. Here, a patient dashboard 328 may be presented to a patient when they log into their account. Patient dashboard 328 may provide a number of applications that utilize the data 326 provided by data sources 300, these applications including, but not limited to, applications for viewing medications, contacting a doctor, setting appointments, setting treatment goals, tracking progress, online exercises, and emergency contacts.

A clinician dashboard 330 may be presented to a clinician when they log into their account. Clinician dashboard 330 may provide a number of applications that utilize the data 326 provided by data sources 300, these applications including, but not limited to, applications for creating intake forms, emergency contacts, contacting clients or staff, adding or registering providers, tracking patient data, creating or managing calendars, and other forms related to a clinical specialty.

An administrator dashboard 332 may be presented to an administrator when they log into their account. Administrator dashboard 332 may provide a number of applications that utilize the data 326 provided by data sources 300, these applications including, but not limited to, applications for assigning a clinician to patients, communicating with doctors, communicating with patients, creating or managing calendars, creating intake forms, and billing.

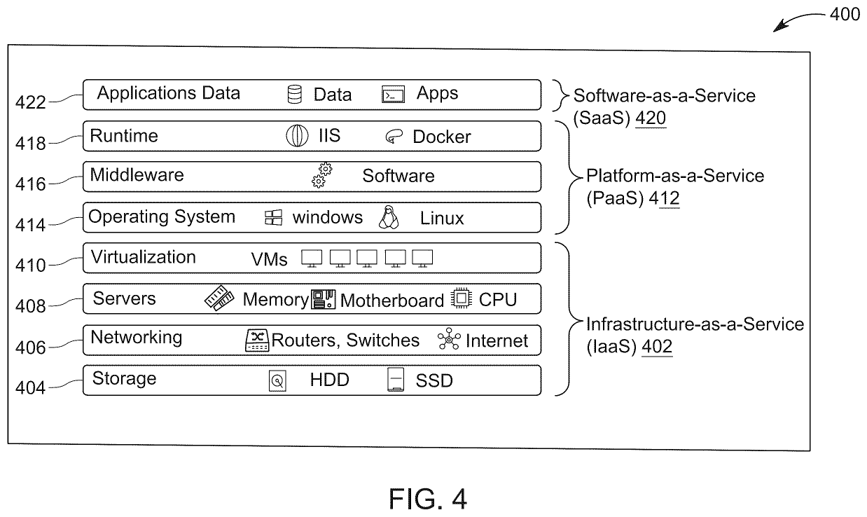

FIG. 4 is a diagram illustrating an exemplary Software-as-a-Service (SaaS) platform suitable for managing healthcare workflows according to an embodiment of the subject matter described herein. Referring to FIG. 4, functionality 400 can be logically divided into layers. Layers may be ordered from least to greatest abstraction of underlying physical resources. Layers may also be divided into groups based on common features for simplicity when referring or billing functions associated with each group.

Infrastructure 402 includes storage function 404, networking function 406, server function 408, and virtualization function 410. Infrastructure functions 404-410 may be bundled together and provided to one or more tenants as a service, referred to as Infrastructure-as-a-Service (IaaS). IaaS is made up of a collection of physical and virtualized resources that provide consumers with the basic building blocks needed to run applications and workloads in the cloud.

Storage function 404 provides storage of data without requiring the user or tenant to be aware of how this data storage is managed. Three types of cloud storage that may be provided by storage function 404 are block storage, file storage, and object storage. Object storage is the most common mode of storage in the cloud because it is highly distributed (and thus resilient), data can be accessed easily over HTTP, and performance scales linearly as the storage grows.

Networking function 406 in the cloud is a form of software defined networking in which traditional networking hardware, such as routers and switches, are made available programmatically, typically through APIs.

Server function 408 refers to various physical hardware resources associated with executing computer-readable code that is not otherwise part of the virtualized network resources in networking function 406 or storage function 404. IaaS providers manage large data centers, typically around the world, that contain the servers powering the various layers of abstraction on top of them and that are made available to end users. In most IaaS models, end users do not interact directly with the physical infrastructure (e.g., memory, motherboard, CPU), but it is provided as a service to them.

Virtualization function 410 provides virtualization of underlying resources via one or more virtual machines (VMs). Virtualization relies on software to simulate hardware functionality and create a virtual computer system. A virtual computer system is known as a “virtual machine” (VM): a tightly isolated software container with an operating system and application inside. Each self-contained VM is completely independent. Putting multiple VMs on a single computer enables several operating systems and applications to run on just one physical server, or “host.” A thin layer of software called a “hypervisor” decouples the virtual machines from the host and dynamically allocates computing resources to each virtual machine as needed. Providers manage hypervisors and end users can then programmatically provision virtual “instances” with desired amounts of compute and memory (and sometimes storage). Most providers offer both CPUs and GPUs for different types of workloads.

Platform 412 includes operating system function 414, middleware function 416, and runtime function 418. Infrastructure functions 404-410 and platform functions 414-418 may be bundled together and provided to one or more tenants as a service, referred to as Platform-as-a-Service (PaaS). In the Platform-as-a-Service (PaaS) model, developers rent everything needed to build an application, relying on a cloud provider for development tools, infrastructure, and operating systems.

Operating system function 414 refers to a PaaS vendor providing and maintaining the operating system that developers use, and the application runs on. For example, Windows and Linux operating systems may be installed in virtual machines and Windows or Linux applications may be run within the operating system.

Middleware function 416 is software that sits in between user-facing applications and the machine's operating system. For example, middleware may allow software to access input from the keyboard and mouse. Middleware is necessary for running an application, but end users don't interact with it. Relatedly, middleware function 416 may also include tools that are necessary for software development, such as a source code editor, a debugger, and a compiler. These tools may be offered together as a framework.

Runtime function 418 is software code that implements portions of a programming language's execution model. A runtime system or runtime environment implements portions of an execution model. Most programming languages have some form of runtime system that provides an environment in which programs run. This environment may address issues including the management of application memory, how the program accesses variables, mechanisms for passing parameters between procedures, interfacing with the operating system, and otherwise. The compiler makes assumptions depending on the specific runtime system to generate correct code. Typically, the runtime system will have some responsibility for setting up and managing the stack and heap, and may include features such as garbage collection, threads or other dynamic features built into the language.

Software 420 includes applications and data function 422. Infrastructure functions 404-410, platform functions 414-418, and software function 422 may be bundled together and provided to one or more tenants as a service, referred to as Software-as-a-Service (SaaS). Applications and data function 422 is the application and associated data created and managed by the user. For example, an application programmed by the user for provided certain functionality disclosed herein may be exposed to the end user via a front end interface such as a web browser or dedicated front end client application. Neither the front end user nor the back end developer is required to manage or maintain services provided by platform 412 and infrastructure 402. This contrasts with on-site hosting of the same functionality.

### Intake

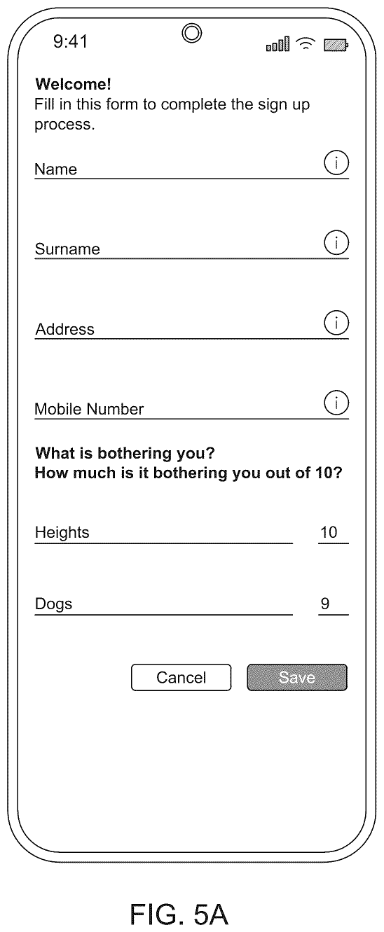

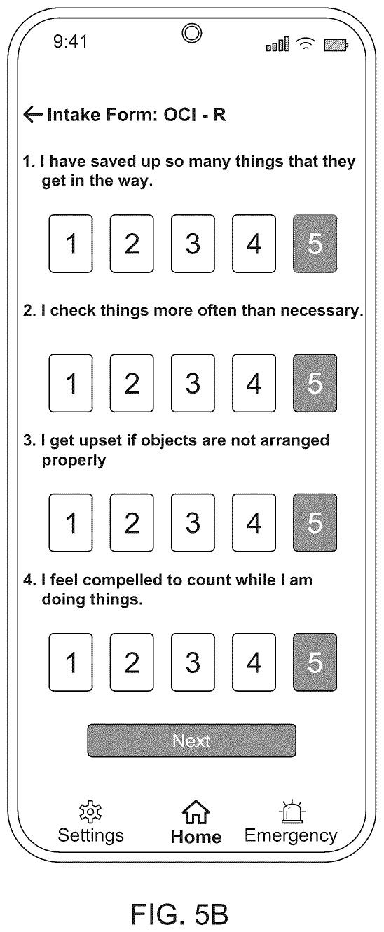

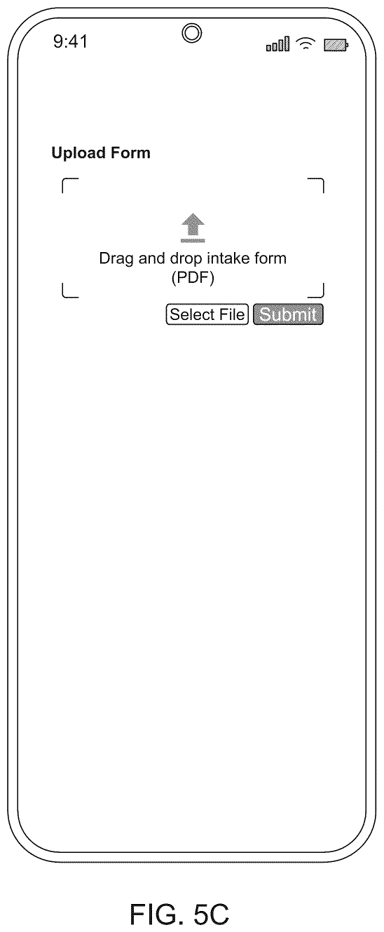

The client onboarding process begins with intake forms designed to collect essential information while ensuring ease of use. FIGS. 5A, 5B, and 5C are diagrams illustrating exemplary user information intake forms for managing healthcare workflows according to an embodiment of the subject matter described herein. FIGS. 5A through 5C illustrate different user information intake forms integral to the system. FIG. 5A shows a client welcome form requesting the client's name, address, mobile number, and a brief description of what is bothering them (e.g., heights, dogs). FIG. 5B shows a more detailed intake form requesting patient medical history and family mental health history. FIG. 5C shows an alternative embodiment where the user can upload an image or scan of an intake form completed on paper, thereby digitizing the information.

FIG. 5A depicts a welcoming client form that requests basic details like the client's name, address, and mobile number. This initial form also includes a space for the client to briefly describe the primary concern bringing them to therapy, allowing for immediate personalization. FIG. 5A shows a client welcome form that prompts users to input their basic details, such as name, address, mobile number, and a brief description of their primary concern (e.g., “heights,” “dogs”). The form is visually divided into sections with clearly labeled fields for easy navigation.

FIG. 5B presents a detailed intake form that gathers a comprehensive medical and family mental health history. This detailed information aids in accurate diagnosis and personalized treatment planning. Recognizing the potential inconvenience of digital-only forms. FIG. 5B shows a more comprehensive intake form that collects medical history and family mental health history. This form includes predefined sections for input, such as “Existing Conditions,” “Medications,” and “Family History.” It is structured to capture both quantitative and qualitative information, including through dropdown menus or text fields.

Referring to FIG. 5B, it may be appreciated that questions may be presented clearly, and the patient can select a response between 1 and 5 depending on how much they agree with the statement. For example, subjective compulsion data may be received from the patient indicating that the patient strongly feels compelled to count while they are doing things (see Question 4). These numbered responses may be aggregated and analyzed by the system and/or the clinician.

FIG. 5C illustrates the flexibility of the system. Clients have the option to upload scanned copies of pre-filled paper forms. This feature ensures inclusivity and accommodates clients who might be more comfortable with traditional methods while simultaneously achieving digitization for efficient data management and secure storage. FIG. 5C presents an alternative interface allowing users to upload images or scanned versions of physical forms. A file upload button with options for selecting files from a local device is displayed. Each form is designed to standardize data collection while accommodating variations in user input methods, including direct typing or document upload.

### Client-Side

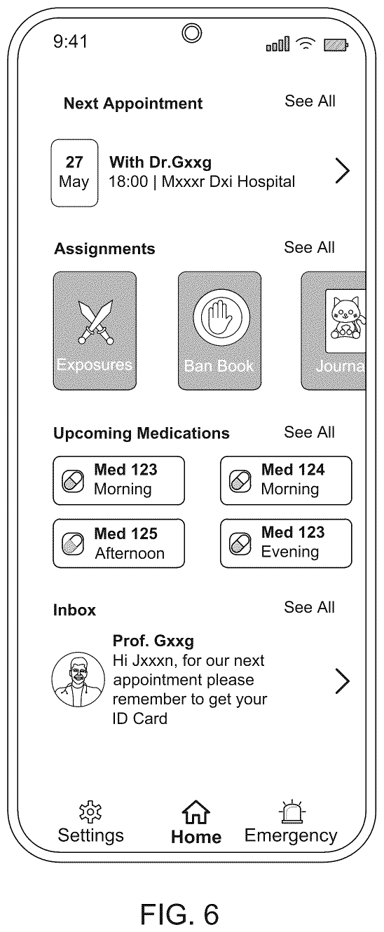

FIG. 6 is a diagram illustrating an exemplary home screen interface for managing healthcare workflows according to an embodiment of the subject matter described herein. Here, details of the patient's next appointment (hospital, office, or virtual-address or Zoom Link) is shown. Next, assignments such as exposures, ban book, and journal assignments are shown. Upcoming medication information is also shown, including an icon and name of each medication dose and the time for each dose. Finally, an inbox is shown for communicating with the doctor and other members of the patient's care team.

FIG. 6 presents a home screen interface tailored to patient users. At the top, a highlighted section displays details about the patient's next appointment, including the date, time, location, and modality (e.g., in-person or virtual, with links for virtual appointments). Below the appointment section, task-based modules for “Exposures,” “Ban Book,” “Journal Assignments,” and “Medication Information” are arranged in a clear grid layout. Each module provides a brief summary or progress indicator, such as “1/3 assignments completed” or medication icons with dosages and scheduled times. Additionally, a communication section labeled “Inbox” allows users to view and send messages to their care team. The layout is designed to consolidate essential patient activities into a single view, with each component visually separated by borders or shaded areas for clarity.

Upon successful onboarding, the client is greeted with a well-organized home screen or dashboard. This central hub, depicted in FIG. 6, provides a clear overview of essential information, empowering clients to actively participate in their treatment journey. Key elements of the dashboard include the details of their next appointment, whether it's an in-person visit to a hospital or office, a virtual session, or a combination of both. The dashboard presents this information clearly, specifying the location, address, or Zoom link, as appropriate. Below the appointment details, the dashboard neatly displays a categorized list of assignments. These assignments are divided into distinct sections for Exposures, Ban Book entries, and Journal prompts, providing a structured approach to therapy. The inclusion of upcoming medication reminders further enhances the dashboard's functionality. Each reminder features a visual icon representing the medication, the name and dosage of the medication, and the specific time for the next dose. This visual and informative presentation helps clients manage their medication schedule effectively. Finally, the dashboard includes a dedicated inbox for seamless communication with the care team. This inbox acts as a centralized hub for messages, facilitating open dialogue and fostering a collaborative approach to treatment.

### Client-Side: Medications

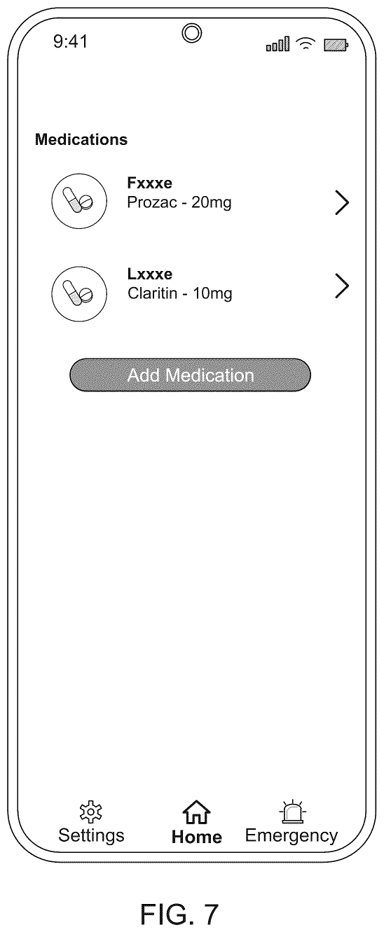

FIG. 7 is a diagram illustrating an exemplary user interface for displaying medication information as part of managing healthcare workflows according to an embodiment of the subject matter described herein. Here, the patient has been prescribed Prozac 20 mg and Claritin 10 mg. Clicking on each medication lets the patient see or edit the information. An Add Medication button allows the patient to add a medication to the list.

FIG. 7 focuses on the medication management interface. The screen lists prescribed medications, each entry displaying the drug name (e.g., Prozac 20 mg), dosage, and timing information. Clicking on a medication opens a detailed view where patients can edit details such as dosage or frequency. A conspicuously placed “Add Medication” button allows users to input new prescriptions by selecting drug names, dosages, and schedules from predefined fields or dropdown menus. A simple, linear layout ensures that users can quickly navigate and manage their medication records.

### Client-Side: Assignments

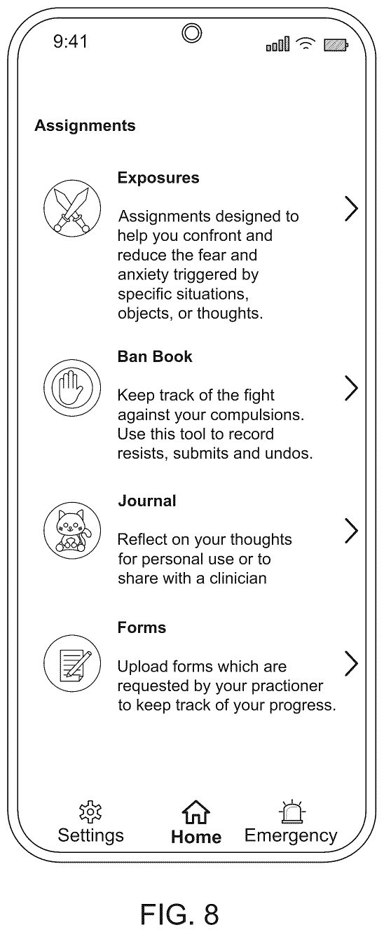

FIG. 8 is a diagram illustrating an exemplary user interface for displaying assignment information as part of managing healthcare workflows according to an embodiment of the subject matter described herein. Here, Exposures are assignments designed to help the user confront and reduce the fear and anxiety triggered by specific situations, objects, or thoughts. Ban Book keeps track of the fight against the user's compulsions. This tool can be used to record resists, submits, and undos. Journal lets the user reflect on their thoughts in writing. Journal entries can be kept private for personal use or may be shared with a clinician. Forms allows the user to upload forms requested by a practitioner to keep track of the patient's progress.

The Assignments section, visualized in FIG. 8, employs a structured approach to therapy, significantly contributing to its effectiveness. By categorizing tasks into four distinct sections-Exposures, Ban Book, Journal, and Forms—the interface provides clarity and encourages client engagement. This clear division ensures clients can easily understand and navigate their treatment plan, promoting a sense of control and ownership over their progress. This structured approach helps break down the often-overwhelming aspects of ERP therapy into manageable steps, facilitating gradual progress and reducing the potential for discouragement.

FIG. 8 showcases an interface for managing therapeutic assignments. The screen is divided into sections for “Exposures,” “Ban Book,” “Journal,” and “Forms.” Each section displays summary details such as the number of completed tasks (“2/5 completed”) or the latest journal entry timestamp. “Exposures” lists ongoing therapeutic tasks aimed at anxiety triggers, while “Ban Book” tracks patient efforts in resisting compulsions. The “Journal” section provides options for creating or reviewing journal entries, and the “Forms” section allows for the uploading of requested documentation. Each module includes action buttons like “View,” “Edit,” or “Upload” for straightforward interaction.

### Client-Side: Exposures

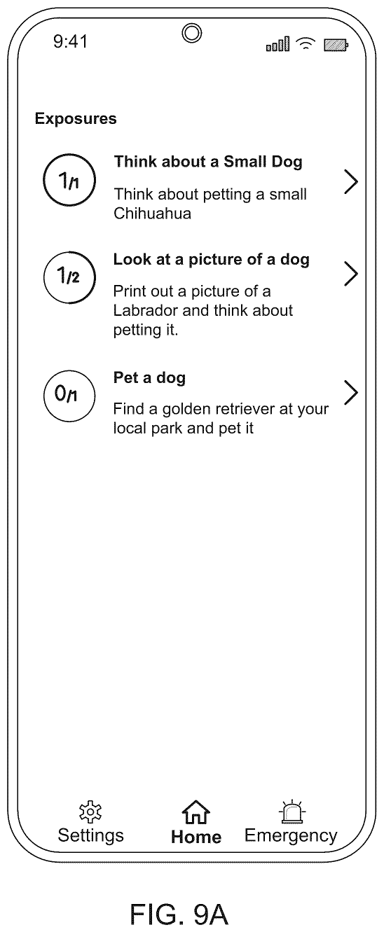

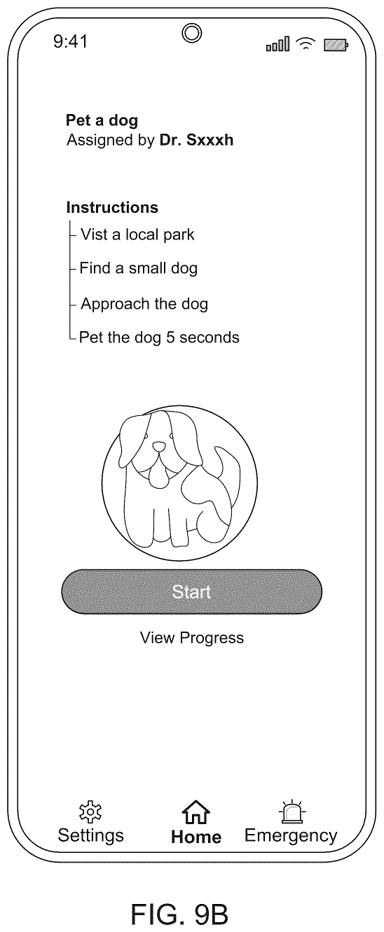

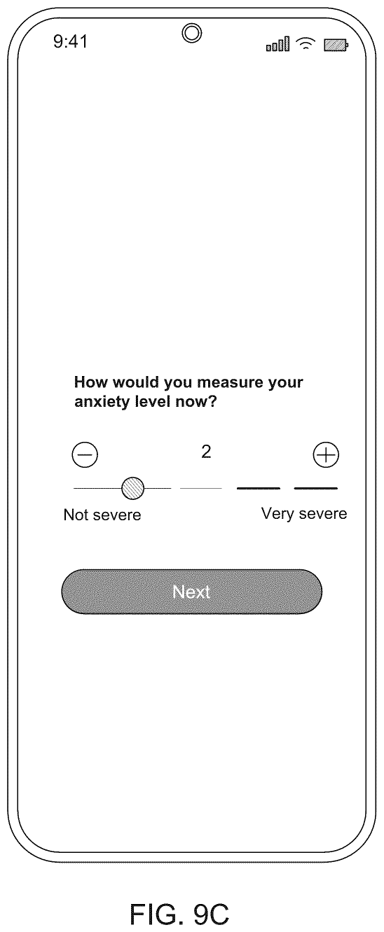

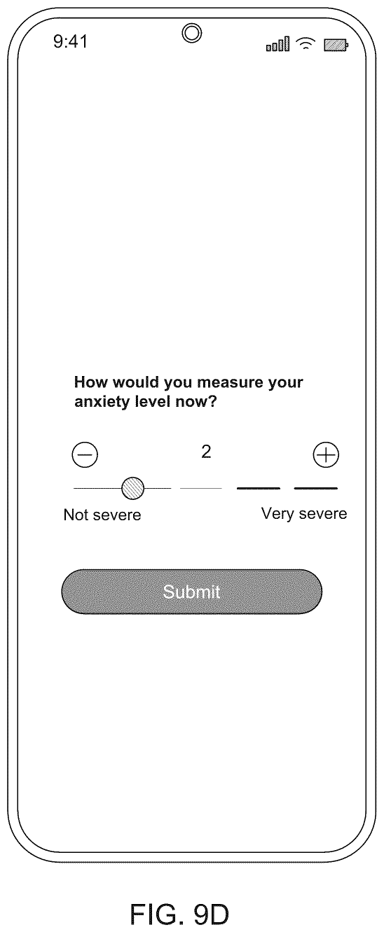

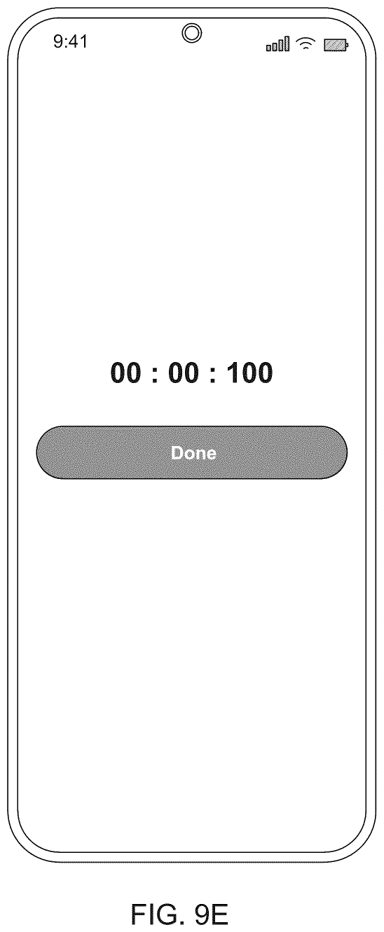

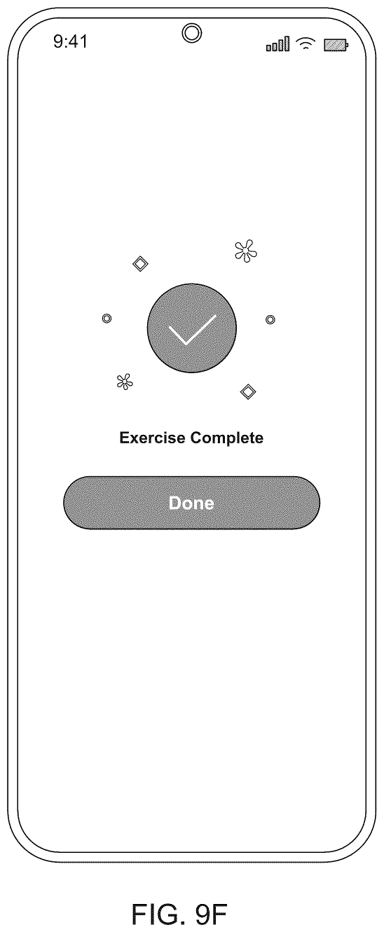

FIGS. 9A to 9F illustrate the Exposures section of the client interface, showcasing its user-friendly design and powerful functionality in guiding clients through their assigned exposures. The interface presents a list of exposure assignments, each designed to help the client gradually confront and reduce the fear and anxiety triggered by specific situations, objects, or thoughts. The example presented focuses on a fear of dogs, a common phobia, and demonstrates how the system breaks down the exposure process into manageable steps.

FIGS. 9A and 9B are diagrams illustrating exemplary user interfaces for presenting a list of assigned exposures and displaying instructions for a selected exposure, respectively, according to an embodiment of the subject matter described herein. Thus, FIGS. 9A and 9B depict the exposure task interface, where patients confront anxiety triggers.

FIG. 9A displays a list of exposures, including “Think about petting a dog,” “Look at a picture of a dog and think about petting it,” and “Find a dog and pet it,” demonstrating a gradual progression in exposure intensity. To ensure clarity and minimize any potential ambiguity, each exposure assignment can include one or more detailed steps. The list view in FIG. 9A shows tasks categorized by difficulty or sequence, such as “Think about a dog,” “View a picture of a dog,” and “Pet a dog.” Each task includes a progress bar or completion status indicator.

FIG. 9B provides a detailed view of an individual task, including step-by-step instructions. FIG. 9B provides a closer look at the instructions for the “Pet a dog” exposure. The assignment is broken down into four clear directives: “Visit a local park,” “Find a small dog,” “Approach the dog,” and “Pet the dog for five seconds.” This meticulous breakdown into specific actions ensures that the client understands the expectations of each exposure step, maximizing its effectiveness. The system intelligently tracks the client's progress through these exposures, automatically noting completed and pending tasks. In the provided example, the system displays progress indicators like “1/1 Think about a small dog exposures completed,” signifying full completion of that particular exposure. Similarly, “1/2 Look at a picture of a dog exposures completed” indicates partial progress, motivating the client towards completion. Finally, “0/1 Pet a dog exposures completed” clearly signifies a pending task, prompting the client to take action. This automated tracking system provides both the client and the clinician with valuable insights into the client's engagement and response to therapy.

FIGS. 9C to 9F show a unique feature integrated into the Exposures section: the ability for clients to log their anxiety levels before and after each exposure task. This real-time tracking of anxiety levels is a crucial aspect of ERP therapy, allowing for precise monitoring of the client's emotional response and facilitating informed adjustments to the treatment plan.

In other words, FIGS. 9C-9F illustrate an anxiety tracking sequence where patients can input an initial anxiety level on a slider or numerical scale, activate a timer during the task, and log their post-task anxiety level upon completion. Visual elements such as timers, progress trackers, and sliders ensure intuitive input and tracking. The exposure assignments are designed to help the patient confront and reduce the fear and anxiety triggered by a fear of dogs. Exposure assignments may include thinking about petting a dog, looking at a picture of a dog and thinking about petting it, and finding a dog and petting it. Exposure assignments may also include one or more detailed steps. For example, the assignment to pet a dog may be broken down into four instructions: visit a local park, find a small dog, approach the dog, and pet the dog for five seconds. Progress may be automatically tracked (e.g., 1/1 Think about a small dog exposures completed, 1/2 Look at a picture of a dog exposures completed, and 0/1 pet a dog exposures completed).

FIGS. 9C, 9D, 9E, and 9F also illustrate an exemplary user interface sequence for tracking a user anxiety level before and after completing an assigned exposure task as part of managing healthcare workflows according to an embodiment of the subject matter described herein. Here, the user may initially report a Not Severe anxiety level of 2. The user can then see and start a timer while they perform the assigned exposure and click Done to stop the timer. Afterward, the user again reports an anxiety level of 2 and the exercise is complete.

FIG. 9C illustrates the initial step, where the user self-reports their anxiety level using a visual scale. In this example, the client reports a “Not Severe” anxiety level of 2.

FIG. 9D shows the initiation of a timer, a tool designed to ensure the client spends an adequate amount of time engaged in the exposure task. As the timer runs, the client actively participates in the assigned exposure. Upon completion, the client clicks the “Done” button to stop the timer, marking the end of the exposure activity.

FIG. 9E displays the post-exposure anxiety level reporting. In this example, the client maintains a consistent anxiety level of 2, indicating successful management of anxiety during the exposure task. This data point is crucial for both the client and the clinician, as it demonstrates progress and reinforces the effectiveness of the exposure therapy.

FIG. 9F signifies the completion of the exposure exercise, highlighting the cyclical nature of this process. This repetition, guided by real-time anxiety level tracking, forms the core of ERP therapy, enabling clients to gradually desensitize themselves to their fears and regain control over their emotional responses.

### Client-Side: Journal

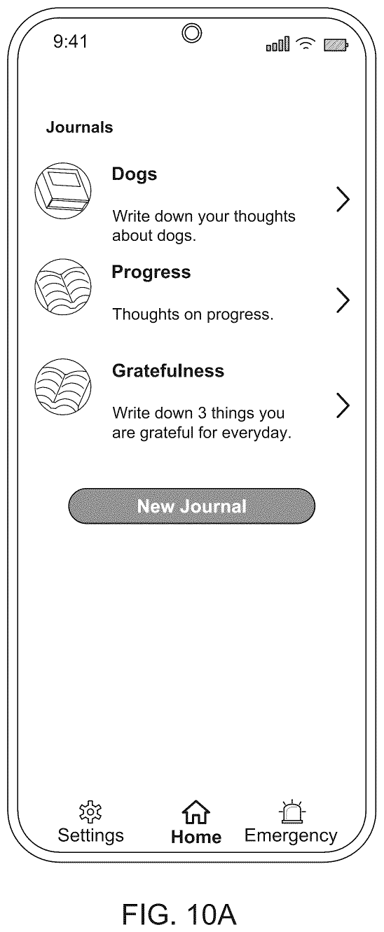

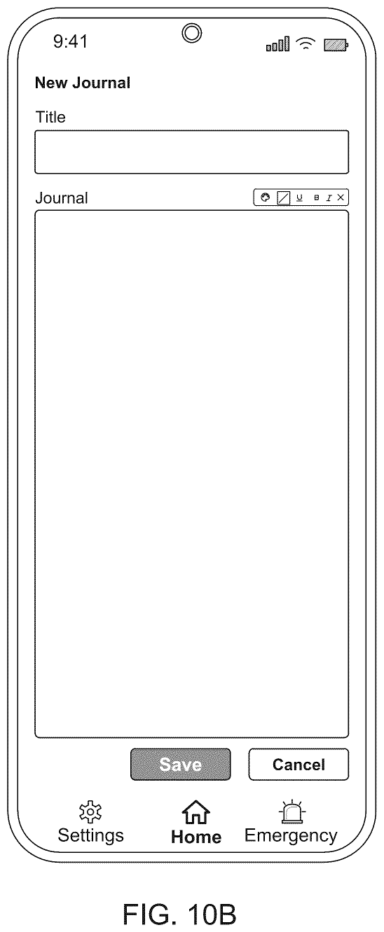

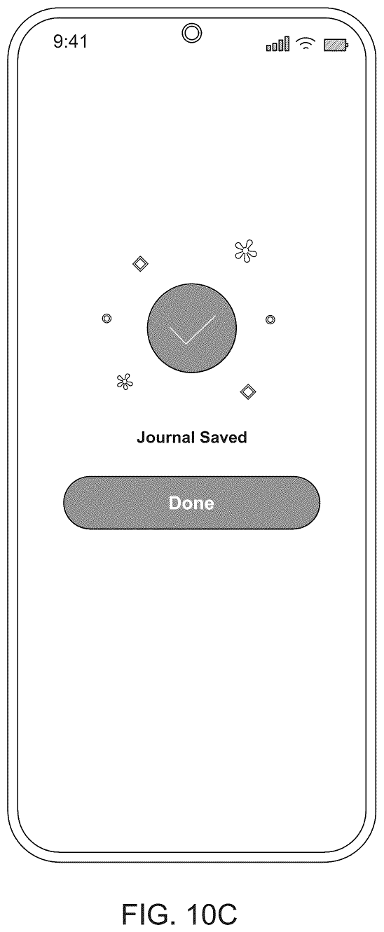

FIGS. 10A, 10B, and 10C are diagrams illustrating exemplary user interfaces for creating, adding, and sharing journal entries as part of managing healthcare workflows according to an embodiment of the subject matter described herein. FIGS. 10A to 10C illustrate the Journal section of the client interface, emphasizing its role in encouraging self-reflection and fostering a deeper understanding of the client's emotional journey. This section provides clients with a dedicated space to document their thoughts, feelings, and experiences related to their therapy. Here, several different types of journals are provided such as a journal relating to fear of dogs, a progress journal, and a gratefulness journal. Once a journal is selected, the user can write an entry in the journal and save when complete. Journals can be automatically stored locally on the user's device and/or synced with a remote data storage device.

FIGS. 10A through 10C illustrate the journaling feature. In FIG. 10A, users select from predefined journal types (e.g., “Fear of Dogs,” “Progress Journal”). FIG. 10B shows the entry creation screen, where users input text into a large editable field. Formatting tools, such as bold or italic text options, may be visible. FIG. 10C presents the journal sharing interface, allowing users to mark entries as “Private” or “Shared with Clinician.” Saved entries are organized by date, with options to edit or delete.

FIG. 10A showcases the variety of journals offered, catering to different aspects of the client's experience. These include a journal specifically related to the fear of dogs, allowing the client to focus on their primary concern; a progress journal to track overall therapeutic progress; and a gratitude journal to encourage positive reflection and cultivate a sense of appreciation. This diverse selection empowers clients to choose a journaling style that best suits their needs and preferences, promoting a sense of ownership and engagement.

FIG. 10B demonstrates the user-friendly interface for creating journal entries. Once a specific journal is selected, the client is presented with a clean and intuitive writing space. They can freely express their thoughts and emotions, allowing for a genuine and unfiltered reflection. Upon completion, the client can save their entry, ensuring its secure storage. The system automatically saves journal entries locally on the user's device, prioritizing privacy and data security.

FIG. 10C shows an optional synchronization feature where clients can choose to sync their journal entries with a remote data storage device, typically a secure, HIPAA-compliant cloud server. This synchronization option allows for data backup and provides an additional layer of security, safeguarding valuable therapeutic insights. It also facilitates easy access to journal entries from different devices, promoting continuity and flexibility in the client's engagement with the system.

### Client-Side: Ban Book

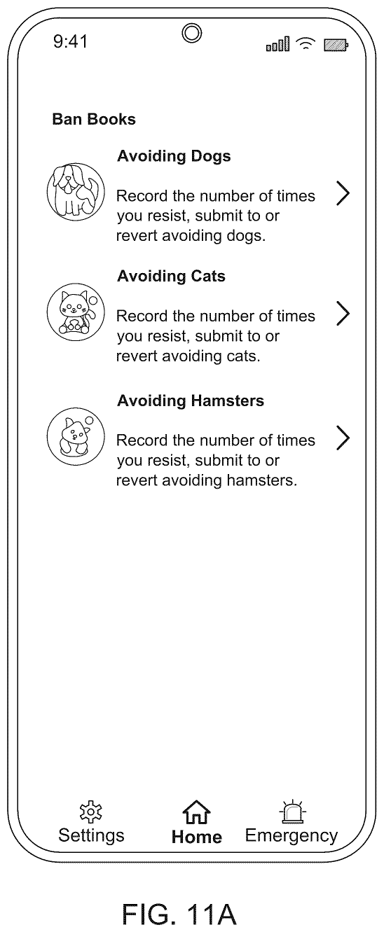

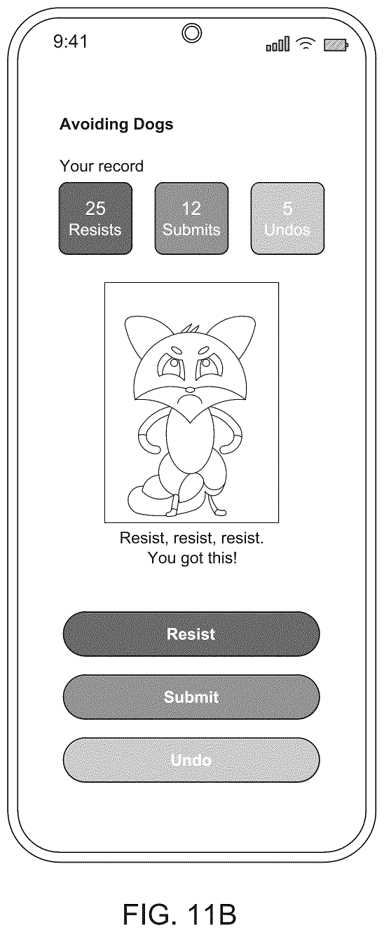

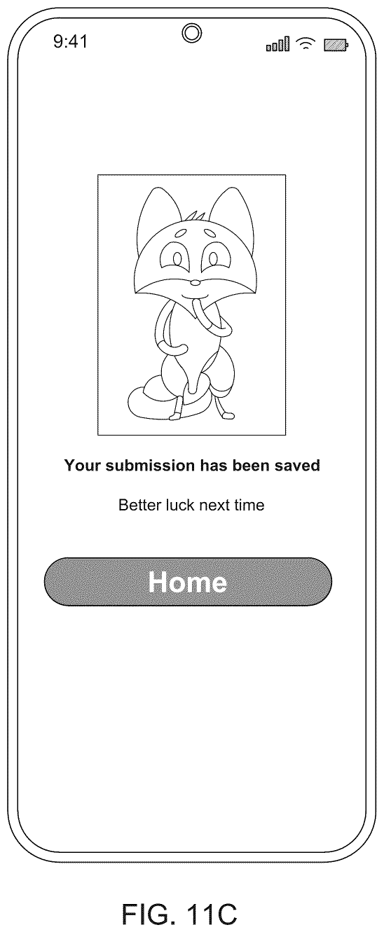

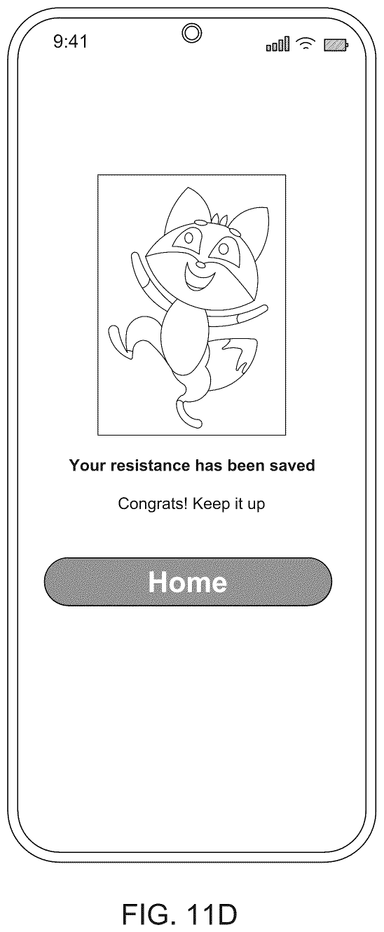

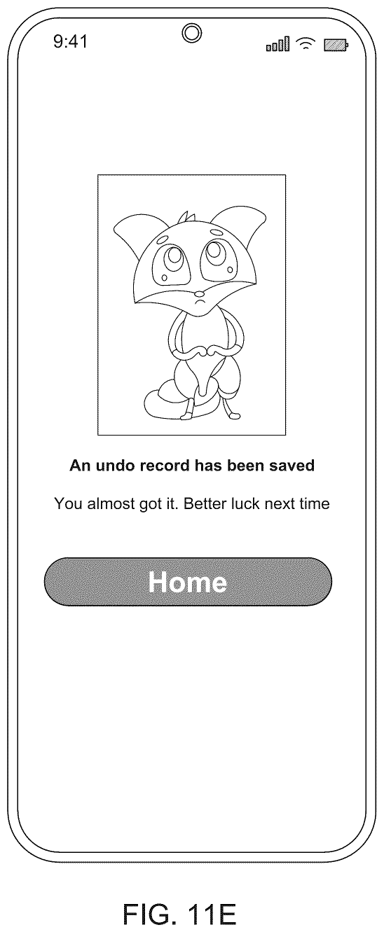

FIGS. 11A to 11E highlight the Ban Book feature, an innovative tool specifically designed to assist clients in managing compulsions and urges that often accompany conditions like OCD.

FIGS. 11A and 11B are diagrams illustrating exemplary user interfaces for selecting and recording progress with ritual resistance as part of managing healthcare workflows according to an embodiment of the subject matter described herein. Here, buttons for resists, submits and undos are colored differently.

FIG. 11A depicts the selection process, where the client chooses a specific ritual resistance category from a list. This categorization allows for focused tracking and analysis of different compulsive behaviors. FIG. 11B focuses on the recording interface, where the client can log their progress in resisting compulsions. The interface features three distinct buttons: “Resists,” “Submits,” and “Undos,” each color-coded for clear differentiation.

FIGS. 11A and 11B display tools for logging ritual resistance. Buttons labeled “Resist,” “Submit,” and “Undo” are distinctly colored and accompanied by tally counts for each action. FIGS. 11C-11E show feedback prompts triggered by user input, such as encouraging messages for successful resistance or neutral prompts for undo actions. Icons and timestamps accompany each log entry, ensuring a detailed and chronological view.

FIGS. 11C, 11D, and 11E are diagrams illustrating exemplary user interfaces for recording a user submission, resistance, and undoing the saving of a record as part of managing healthcare workflows according to an embodiment of the subject matter described herein. Here, example icons and messages are shown that may be displayed to the user in response to a submit, resists, or undo respectively. It is appreciated that different icons and messages may be presented to either encourage or discourage the user, depending on circumstances.

FIGS. 11C, 11D, and 11E provide visual examples of the system's feedback in response to user input. Upon recording a submission to a compulsion, the system may display a specific icon, such as a frowning face, and a corresponding message like “You submitted.” This visual and textual feedback acknowledges the event without judgment, encouraging honest self-reporting. In contrast, recording a successful resistance to a compulsion triggers a positive response.

FIG. 11D shows an example where the system displays a smiling face icon and a message like “You resisted! Great job!” This positive reinforcement aims to motivate the client and acknowledge their efforts in managing their compulsions.

FIG. 11E demonstrates the “Undo” functionality, allowing clients to rectify accidental entries or instances where they might engage in a self-comforting behavior shortly after resisting a compulsion. The system might display a neutral icon, such as a clock, and a message like “You undid your resist,” providing a factual record of the event. This comprehensive tracking system, coupled with visual feedback, empowers clients to gain valuable insights into their compulsive behaviors, recognize patterns, and develop effective strategies to manage their urges. The collected data is invaluable for both the client and the clinician in understanding the nature and frequency of compulsions, enabling personalized treatment adjustments and fostering a sense of progress.

### Client-Side: User Direct Messages

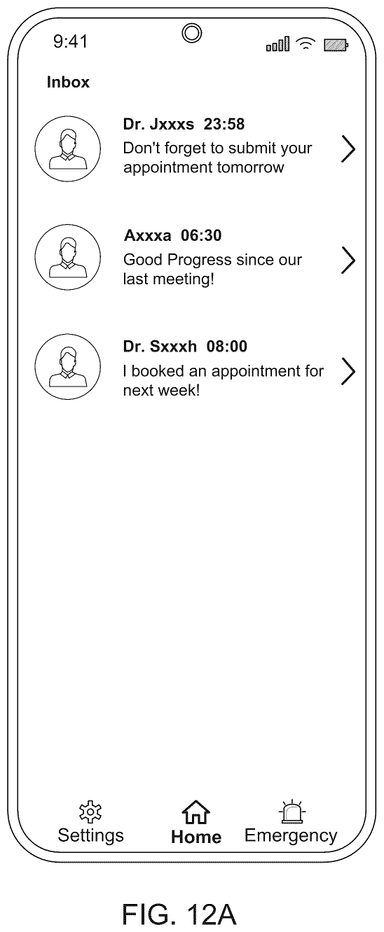

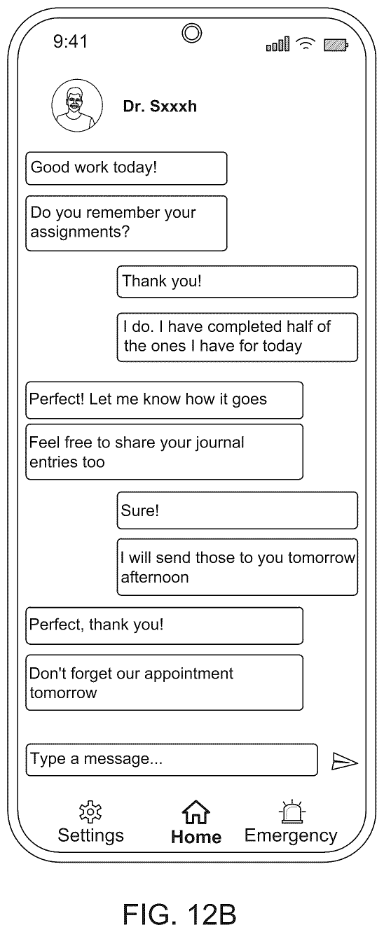

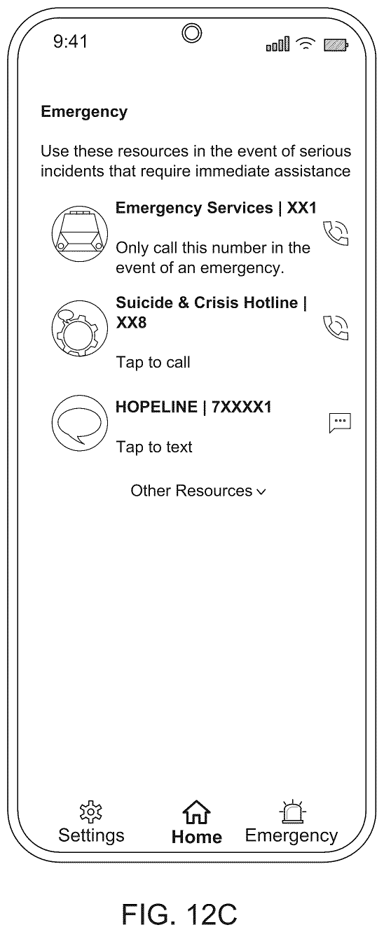

Recognizing the crucial role of continuous support in successful therapy, the client interface integrates secure messaging features designed to facilitate seamless communication between clients and their care team. FIGS. 12A and 12B illustrate the direct messaging interface. FIG. 12A shows a list of doctors who are members of the client's care team. The client can easily select a specific doctor to initiate a conversation.

FIGS. 12A, and 12B illustrate an exemplary user interface (direct messaging interface) for sending and receiving direct messages between users and care team members according to an embodiment of the subject matter described herein. Here, several doctors are members of the user's care team. Clicking on a doctor allows the user to see a communication history with that doctor and direct message them. Users can select a care team member from a list, view past messages, and send new ones via a text input field.

FIG. 12B displays the communication history, allowing both the client and the doctor to review previous messages and maintain context within the conversation. The interface also provides a text box for composing new messages, ensuring easy and intuitive communication.

FIG. 12C is a diagram illustrating an exemplary user interface for emergency services according to an embodiment of the subject matter described herein. Here, various phone numbers and instructions on when to use them are provided for quick and easy access by the user.

FIG. 12C provides emergency contact resources, listing critical phone numbers (e.g., crisis hotlines) with clear labels and usage guidelines. FIG. 12C highlights the Emergency Services section, a crucial element designed to provide immediate assistance in critical situations. This section provides a list of essential phone numbers along with clear instructions on when to use each contact. This instant access to emergency resources ensures that clients have the support they need, promoting a sense of safety and security throughout their therapeutic journey. The inclusion of clear instructions reduces potential confusion during stressful situations, ensuring that clients can quickly and effectively seek appropriate help when needed.

### Clinician-Side

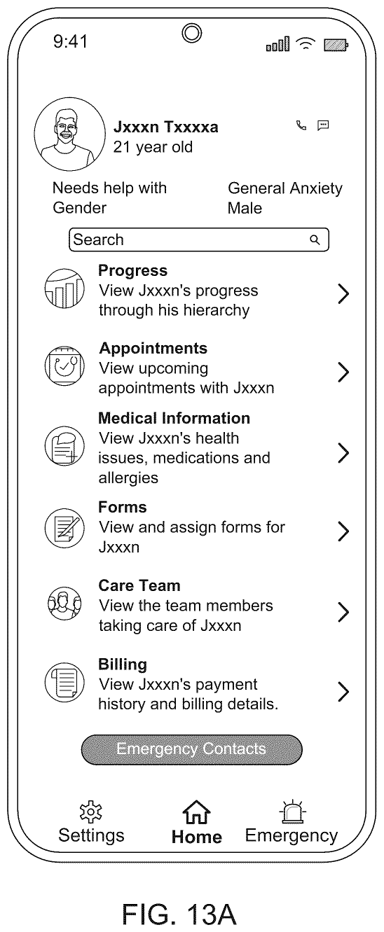

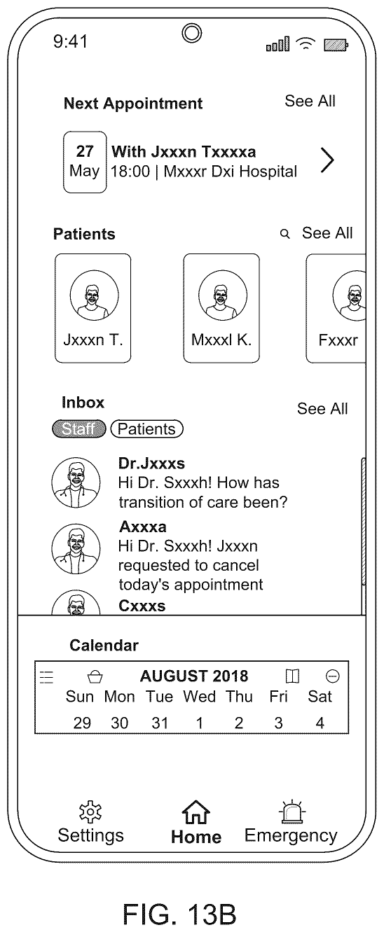

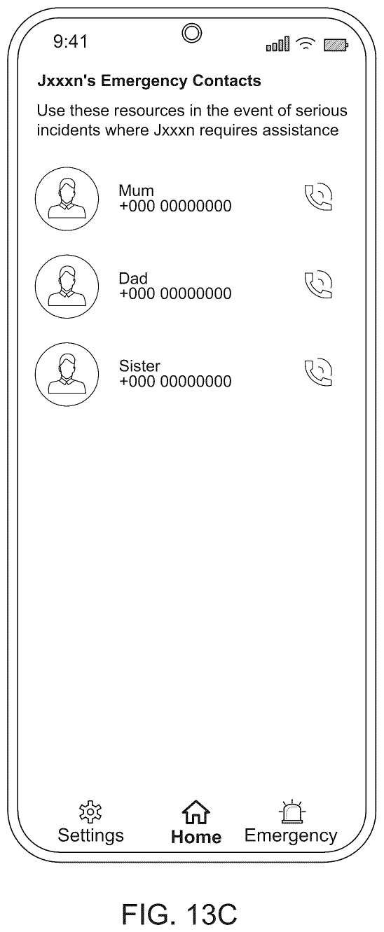

FIGS. 13A through 13C depict the clinician dashboard. FIG. 13A consolidates patient data, including progress metrics, upcoming appointments, and task summaries, into a single view. FIG. 13B organizes appointments by date and patient, displaying details such as time, contact information, and a location map. FIG. 13C highlights an emergency widget listing the patient's emergency contacts with quick access options.

FIG. 13A is a diagram illustrating an exemplary home screen user interface for clinicians according to an embodiment of the subject matter described herein. Here, the clinician can view a patient's progress, appointments, medical information, forms, members of the care team assigned to the patient, and billing information in a single unified home screen.

The clinician-side interface begins with a comprehensive dashboard (FIG. 13A) that provides a holistic view of the patient's journey. This centralized platform streamlines information access for clinicians, enabling them to make informed decisions regarding the patient's treatment plan. Key elements of the dashboard include summaries of the patient's progress in various aspects of their therapy, such as exposures, ban book entries, and journal reflections. This allows clinicians to quickly assess the patient's overall engagement and response to treatment. Information regarding upcoming appointments, including the date, time, and location, is also prominently displayed, ensuring that clinicians can efficiently manage their schedules and ensure timely care for their patients. Access to the patient's medical information, including diagnoses, medication history, and relevant medical conditions, is readily available, allowing clinicians to consider any potential physical health factors that might influence the patient's mental health. The dashboard also includes details about the forms that have been assigned to the patient, allowing clinicians to track completion and monitor progress on standardized assessments. Information about the members of the care team assigned to the patient is readily accessible, facilitating collaboration and ensuring a coordinated approach to treatment. Lastly, the dashboard provides access to the patient's billing information, streamlining administrative processes and ensuring timely management of financial aspects.

FIG. 13B is a diagram illustrating an exemplary appointments user interface for clinicians according to an embodiment of the subject matter described herein. Here, the next appointment with the patient is shown at the top of the interface, a list of other patients associated with the clinician is shown in the middle of the interface, an inbox for viewing messages is shown in a lower part of the interface, and a calendar is shown at the bottom of the interface.

FIG. 13B showcases a dedicated Appointments section, providing a more focused view of the clinician's schedule and patient interactions. The top of the interface displays the details of the next appointment with the selected patient, ensuring that this critical information is readily available. The middle section presents a list of other patients associated with the clinician, enabling quick navigation between different patient profiles. A dedicated inbox for viewing messages is positioned in the lower part of the interface, facilitating seamless communication with patients and other care team members. Finally, a calendar view is displayed at the bottom of the interface, providing a visual representation of the clinician's schedule, appointments, and availability. This comprehensive Appointments section streamlines schedule management and promotes efficient organization for the clinician.

FIG. 13C is a diagram illustrating an exemplary emergency widget user interface for clinicians according to an embodiment of the subject matter described herein. Here, the patient has three emergency contacts including Mum, Dad, and Sister. The clinician can quickly and easily contact these contacts in the event of an emergency with the patient.

FIG. 13C focuses on an emergency widget integrated into the clinician interface, demonstrating the system's commitment to patient safety and timely intervention in critical situations. This widget displays a list of the patient's emergency contacts, including their names and relationships to the patient. In the provided example, the emergency contacts include “Mum,” “Dad,” and “Sister.” This readily accessible information empowers clinicians to quickly reach out to designated individuals in case of an emergency, ensuring that the patient receives appropriate support even outside of scheduled therapy sessions. The emergency widget demonstrates a proactive approach to patient care, recognizing the importance of immediate action in situations that may require intervention beyond the scope of traditional therapy.

### Clinician-Side: Exposures

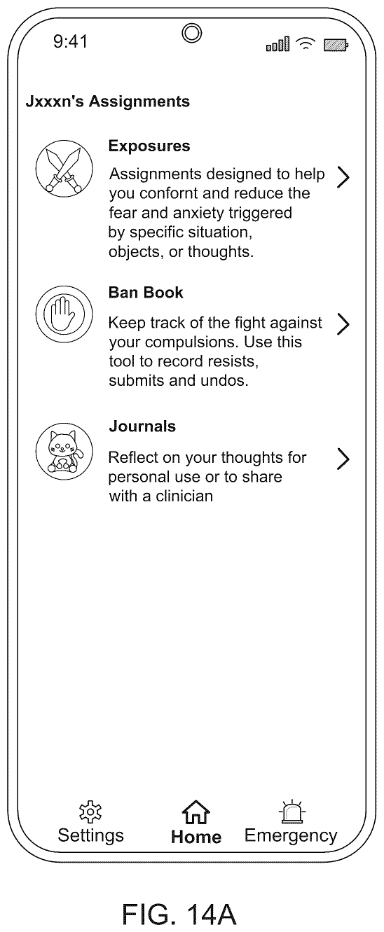

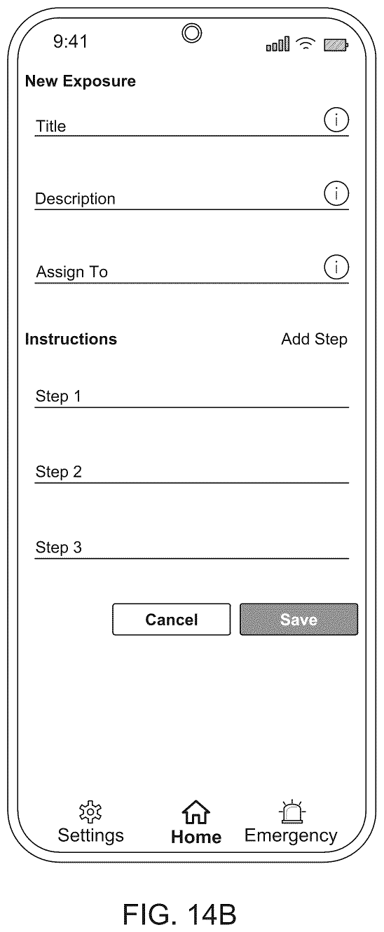

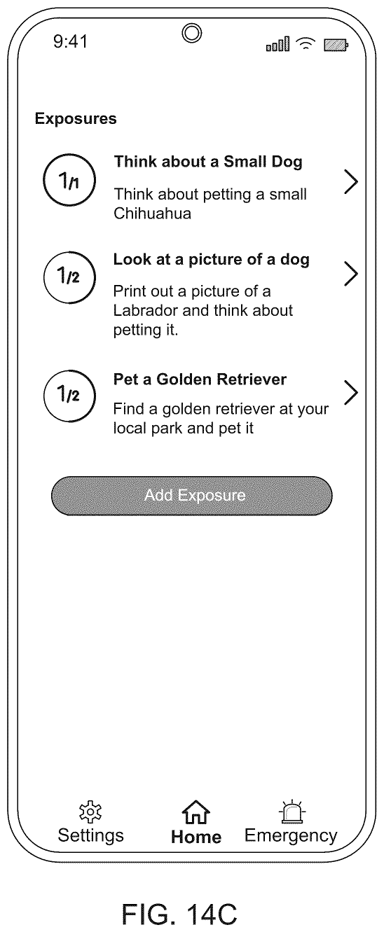

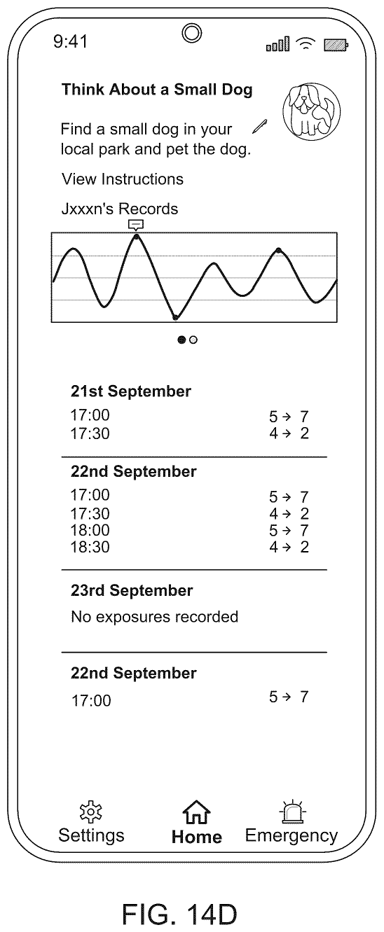

FIGS. 14A, 14B, and 14C are diagrams illustrating exemplary user interfaces for allowing clinicians to view currently assigned exposures, assign new exposures, and archive completed exposures according to embodiments of the subject matter described herein.

FIGS. 14A to 14D illustrate the Exposures management section within the clinician interface, highlighting its role in tailoring exposure therapy to each patient's specific needs. FIGS. 14A and 14B allow clinicians to assign new exposure tasks by specifying task titles, descriptions, and detailed steps.

FIG. 14A displays the interface for assigning new exposures, providing clinicians with the flexibility to create customized tasks for their patients. The interface includes fields for entering a title and a detailed description of the exposure, ensuring clarity and minimizing ambiguity for both the clinician and the patient. The clinician can also specify the user to whom the exposure is assigned and add a series of steps that the patient needs to complete as part of the exposure task. This step-by-step breakdown ensures that even complex exposures are presented in a manageable and understandable format for the patient.

FIG. 14B visualizes the process of archiving completed exposures, allowing clinicians to maintain a clean and organized record of the patient's progress. Archiving completed exposures keeps the active task list focused on current goals while simultaneously preserving a complete history of the patient's journey.

FIG. 14C shows archived tasks categorized by status or date. FIG. 14C provides an alternative view of the exposure assignment process, demonstrating the system's flexibility in accommodating different workflows.

FIG. 14D showcases the system's capability to visually represent the patient's progress on their exposures, empowering clinicians with data-driven insights to inform treatment adjustments. A timeline or chart displaying the patient's progress over time is presented, allowing the clinician to quickly identify periods of success and challenges. Below the visual representation, more detailed information regarding each exposure task is available, providing context and facilitating in-depth analysis of the patient's response to therapy. This combination of visual summaries and detailed information enables clinicians to make informed decisions about the frequency, intensity, and types of exposures assigned, ensuring the treatment plan remains aligned with the patient's evolving needs.

FIG. 14D presents progress data, such as task completion rates over time, in a timeline or chart format. A title and description for a new exposure may be provided, along with the user it is assigned to and any steps associated with the new exposure.

FIG. 14D is a diagram illustrating an exemplary user interface for allowing clinicians to view client progress on exposures according to an embodiment of the subject matter described herein. Here, a timeline or chart of the user's progress is displayed along with more detailed information below. This allows the clinician to quickly and easily see when the patient had the least and most success with their treatment.

### Clinician-Side: Ban Book

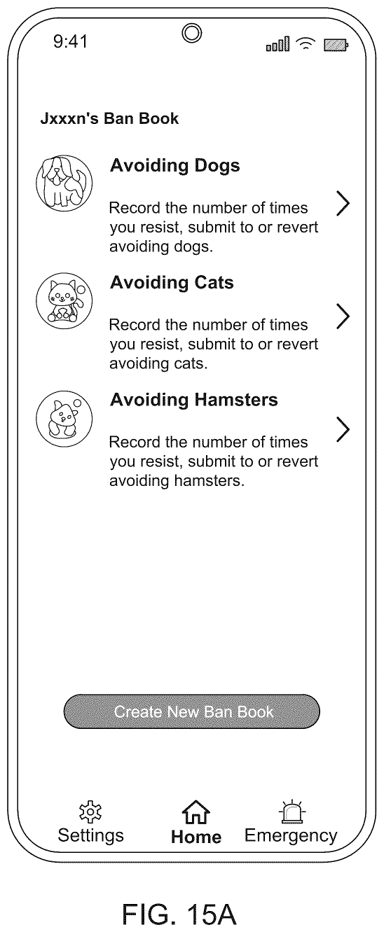

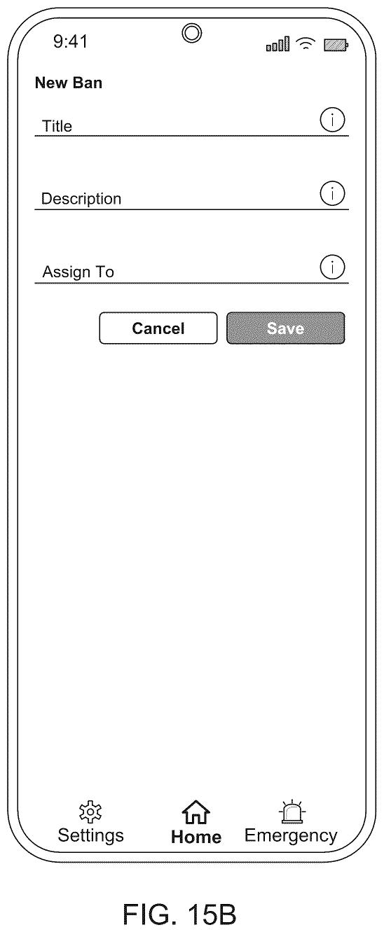

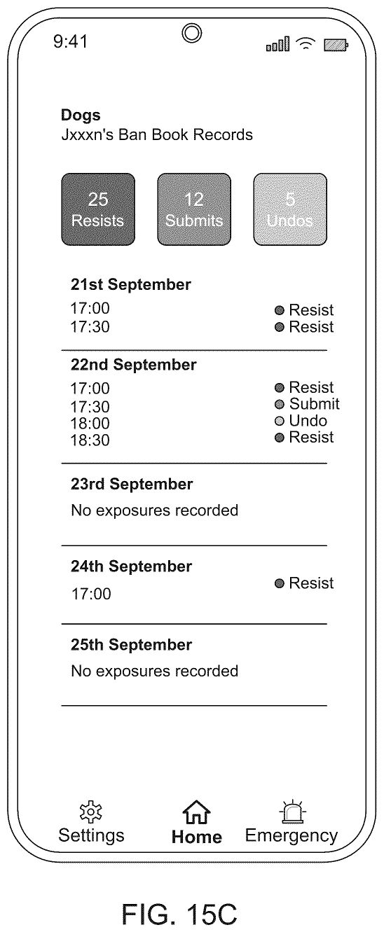

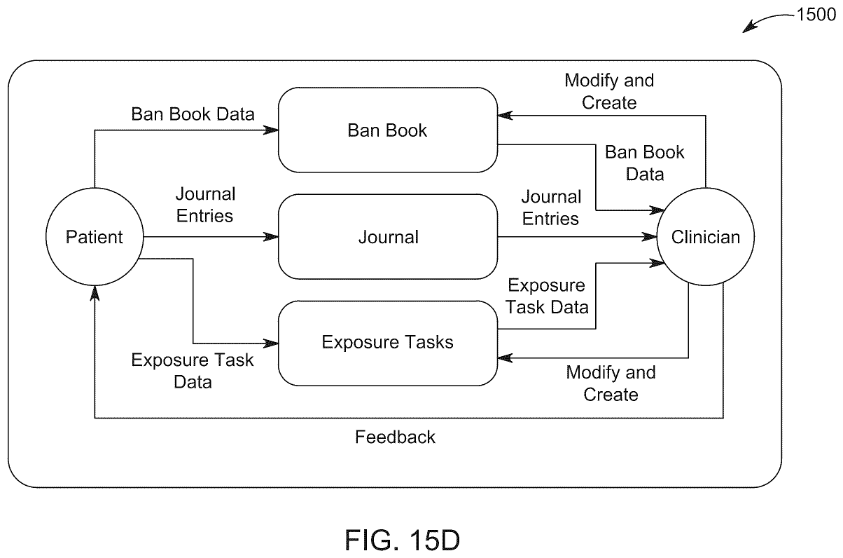

FIGS. 15A and 15B illustrate interfaces for assigning ritual resistance tasks. Each task includes a name, description, and status. FIG. 15C shows progress tracking with color-coded tallies for resists, submits, and undos, alongside detailed logs. FIG. 15D connects this data flow to clinician feedback, enabling real-time updates to treatment plans.

FIGS. 15A to 15C delve into the Ban Book functionality from the clinician's perspective, emphasizing its role in understanding and managing the patient's compulsive behaviors.

FIGS. 15A, and 15B are diagrams illustrating exemplary user interfaces for allowing clinicians to assign new ritual resistance categories and archive completed assignments according to embodiments of the subject matter described herein.

FIG. 15A illustrates the interface for assigning new ritual resistance categories, empowering clinicians to customize the Ban Book to address specific compulsions that the patient is struggling with. FIG. 15B depicts the process of archiving completed assignments, allowing clinicians to maintain a clear record of the patient's progress in managing their compulsions. This archiving function ensures that the active Ban Book remains focused on current challenges while preserving a complete history of the patient's journey.

FIG. 15C is a diagram illustrating an exemplary user interface for allowing clinicians to view client progress on ritual resistances according to an embodiment of the subject matter described herein. Here, a color-coded tally of resists, submits, and undos is shown at the top of the interface. Below, detailed information sorted by date is shown.

FIG. 15C showcases the clinician's view of the patient's progress on their ritual resistances. A color-coded tally of resists, submits, and undos is prominently displayed at the top of the interface, providing a quick visual summary of the patient's success in managing their compulsions. Below this summary, detailed information sorted by date is presented, allowing clinicians to identify patterns and trends in the patient's behavior. This comprehensive data, combined with visual aids, empowers clinicians to assess the effectiveness of interventions, identify potential triggers, and make informed adjustments to the treatment plan to better support the patient's journey towards overcoming their compulsions.

The system described herein acts as an expert-in-the-loop system, where the application collects data and presents it to the clinician (the expert), for example through FIG. 15C, in order to make better judgement based on real data. This will allow the clinician to more efficiently treat the patient, as he can make data-backed decisions, which contrasts with conventional therapy sessions which may be heavily subjective. The clinician will be able to provide better guidance to the patient during therapy sessions, as well as modify and assign new tasks in the app.

FIG. 15D is a diagram illustrating an exemplary feedback process 1500 according to an embodiment of the subject matter described herein. Referring to FIG. 15D, the patient may provide ban book data, journal entries, and exposure task data into functional elements of the application for the ban book, the exposure tasks, the journal, respectively. This information may be provided to the clinician, who can modify or create bank book data and exposure task data. Notably, however, the clinician cannot add or modify patient journal entries, as those are solely provided by the patient. The clinician can also provide feedback to the patient. In this way, the application acts as an information and communications hub between the patient and the clinician.

### Clinician-Side: Journal

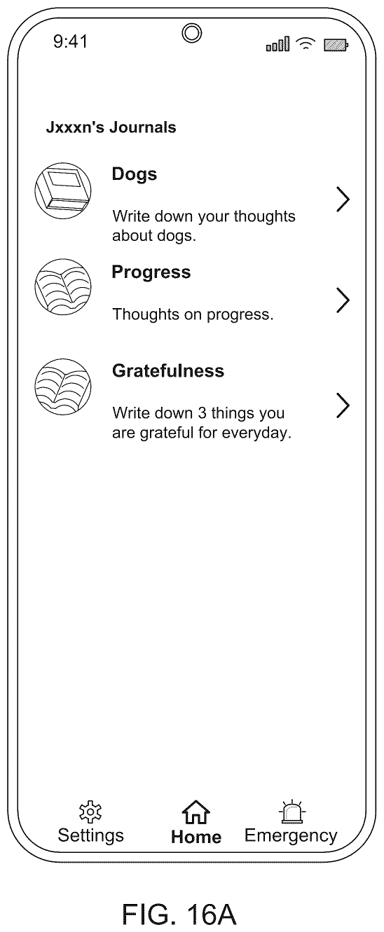

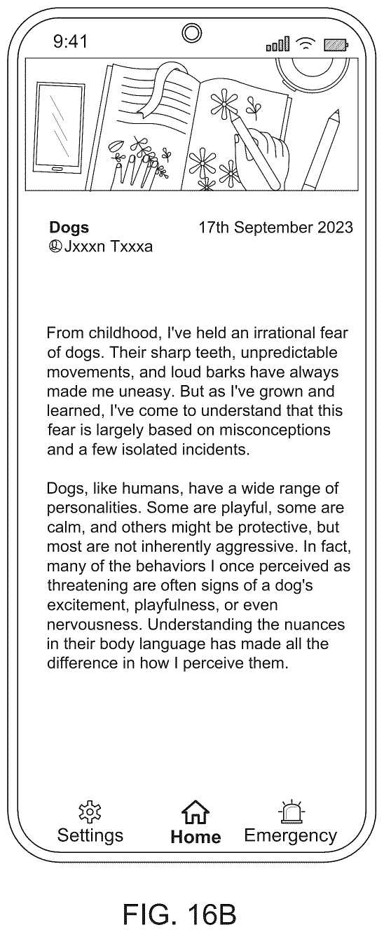

FIGS. 16A and 16B are diagrams illustrating exemplary user interfaces for allowing clinicians to view any journals shared by a client or patient according to embodiments of the subject matter described herein. FIGS. 16A and 16B display clinician-facing journal review tools. Shared entries are organized chronologically and tagged by type or topic (e.g., “Fear of Dogs”). Each entry includes a timestamp and a read/unread indicator. The patient has entered an example journal entry on Sep. 17, 2023 regarding their childhood experiences with dogs. This may only be viewable by the clinician if the patient has chosen to share this information.

FIG. 16A displays a list of journals shared by the client, ensuring that clinicians only have access to entries explicitly shared by the patient. This prioritizes patient privacy and ensures that the client maintains control over the information they choose to disclose with their clinician.

FIG. 16B provides a detailed view of a selected journal entry. In the provided example, the patient has shared an entry dated Sep. 17, 2023, reflecting on their childhood experiences with dogs, which might offer valuable insights into the origins of their current fear. This access to the patient's personal reflections enables clinicians to build a more comprehensive understanding of the individual's experiences, fears, and motivations, facilitating a more empathetic and personalized approach to therapy.

### Clinician-Side: Appointments

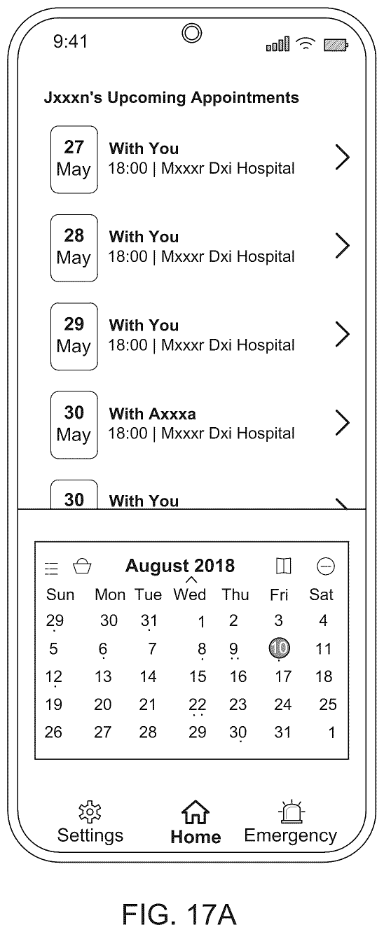

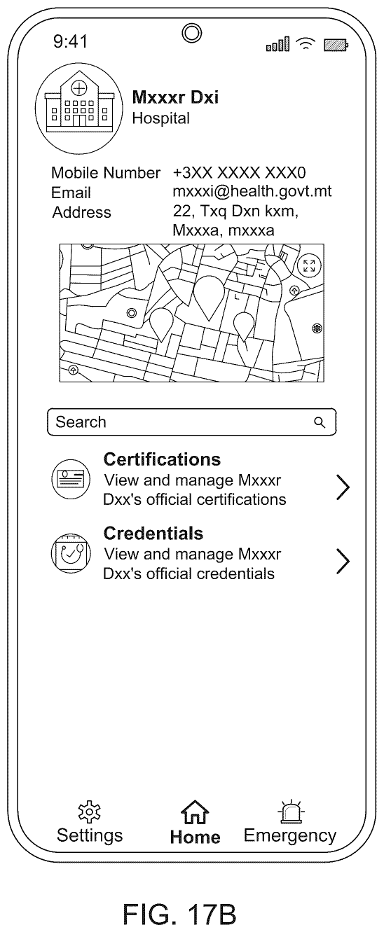

FIGS. 17A and 17B are diagrams illustrating exemplary user interfaces for allowing clinicians to view upcoming and completed appointments, view detailed appointment information, and schedule or cancel appointments according to embodiments of the subject matter described herein. FIGS. 17A and 17B provide an interface for managing appointments. Details such as patient names, contact information, and appointment locations are consolidated into a list or calendar view, ensuring efficient scheduling. All relevant information is shown together, including mobile number, email address, physical address, and visual map of the selected appointment location.

FIG. 17A provides an overview of upcoming and completed appointments, allowing clinicians to manage their schedule and track patient interactions effectively.

FIG. 17B shows the details of a selected appointment, presenting all relevant information in a clear and organized format. This includes the patient's mobile number, email address, physical address of the appointment location, and even a visual map for easy navigation. This comprehensive display of appointment information ensures that clinicians have everything they need to prepare for and conduct successful therapy sessions. Clinicians can directly schedule or cancel appointments through the interface, streamlining the administrative process and promoting efficient time management

### Clinician-Side: Patient Medical Information

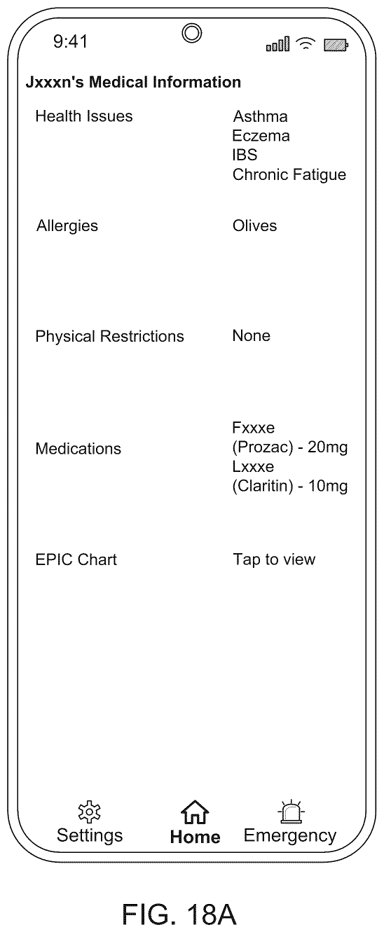

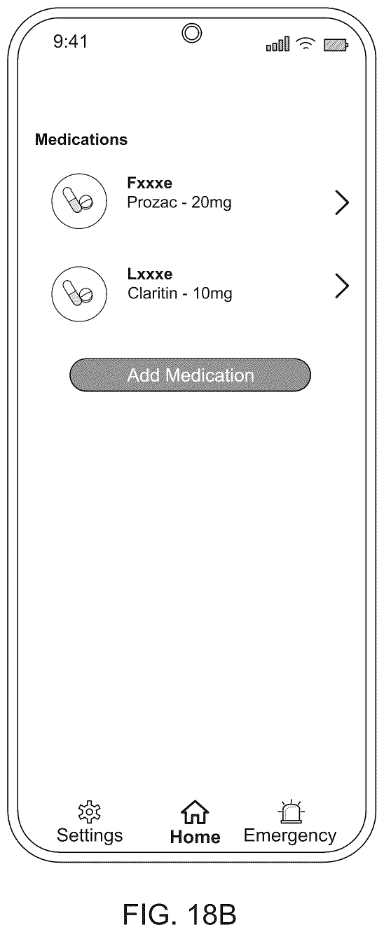

FIGS. 18A and 18B are diagrams illustrating exemplary user interfaces for accessing a patient's medications and medical information according to embodiments of the subject matter described herein. FIGS. 18A and 18B organize patient medical data into categories like “Medications” and “Health Records.” Interactive elements allow clinicians to edit or add information as needed. FIGS. 18A and 18B illustrate the clinician's access to patient medical information and medications.

FIG. 18A shows the interface for accessing the patient's medication list, including details about dosage, frequency, and any potential side effects.

FIG. 18B displays the access point for the patient's general medical information, allowing clinicians to review diagnoses, medical history, and any relevant medical conditions. This comprehensive view of the patient's health profile enables clinicians to consider potential interactions between physical and mental health, ensuring a holistic approach to treatment. It's important to note that the information presented to clinicians might differ from the patient's view, providing a more in-depth and professional perspective on the patient's health records.

### Clinician-Side: Forms

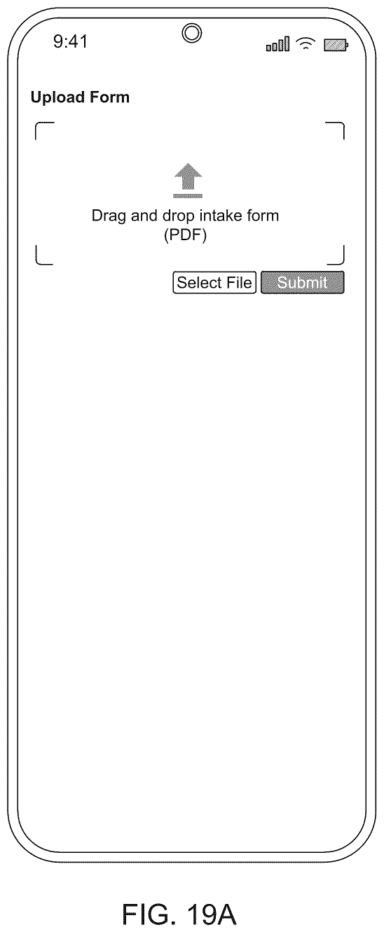

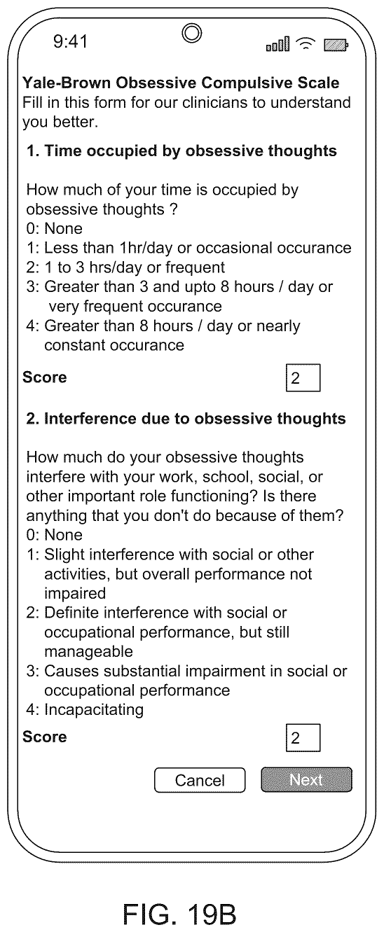

FIGS. 19A and 19B are diagrams illustrating exemplary user interfaces for uploading, creating, or editing forms and assigning forms to patients according to embodiments of the subject matter described herein. FIGS. 19A and 19B show interfaces for handling forms. Users can upload scanned documents or complete digital forms directly, with clear labels indicating form type and status. It is appreciated that the medical information and medications information presented may be different for the clinician than for the patient. Here, the user can either upload an image of a completed form or can complete the Yale-Brown OCD Scale form directly on their device.

FIG. 19A displays a list of available forms, including the Yale-Brown OCD Scale, a widely used assessment tool for OCD. Clinicians can upload images of completed forms, digitizing these records for efficient storage and analysis.

FIG. 19B demonstrates the system's capability to support direct form completion within the interface. This streamlines the assessment process, reducing the reliance on paper-based methods and minimizing potential data entry errors. This integrated form management functionality ensures that clinicians have access to the tools they need for comprehensive evaluation and accurate progress monitoring.

### Clinician-Side: Care Team

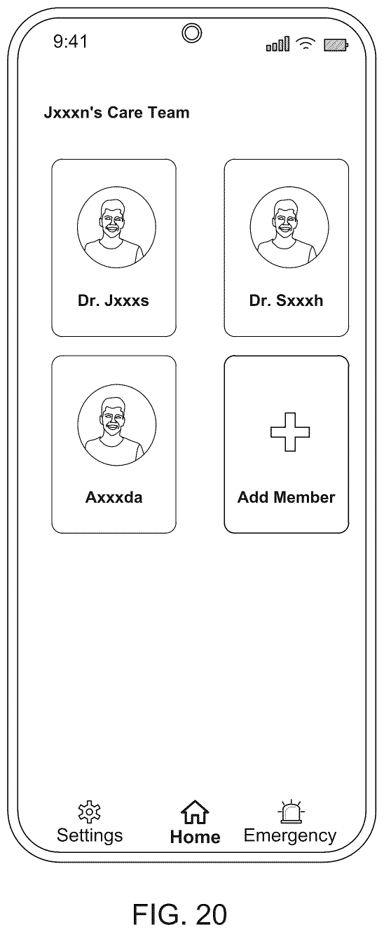

FIGS. 20 through 22 include tools for managing care teams, logging billing details, and accessing emergency resources. Each screen uses a structured layout with labeled sections for easy navigation. FIGS. 20 to 22 highlight the system's support for collaborative care, recognizing the importance of a coordinated approach involving multiple professionals to ensure optimal patient outcomes.

FIG. 20 is a diagram illustrating an exemplary user interface for adding or removing a user account as a member of a client's care team according to embodiments of the subject matter described herein. FIG. 20 shows the Care Team management functionality which allows clinicians to add or remove members from the patient's care team. This ensures that all relevant professionals, such as therapists, psychiatrists, and primary care physicians, have access to the patient's information and can contribute to the treatment plan.

### Clinician-Side: Billing

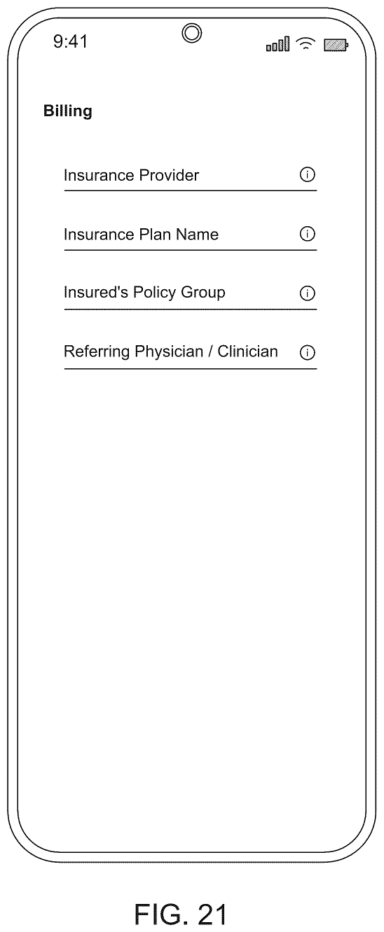

FIG. 21 is a diagram illustrating an exemplary user interface for logging client billing information according to embodiments of the subject matter described herein. FIG. 21 provides a dedicated interface for logging client billing information. This streamlines financial management, enabling clinicians to track services provided, generate invoices, and ensure timely payment for their services. The system's integration of billing functionality emphasizes its practicality and recognizes the importance of efficient administrative processes in a clinical setting.

### Clinician-Side: Emergency Resources

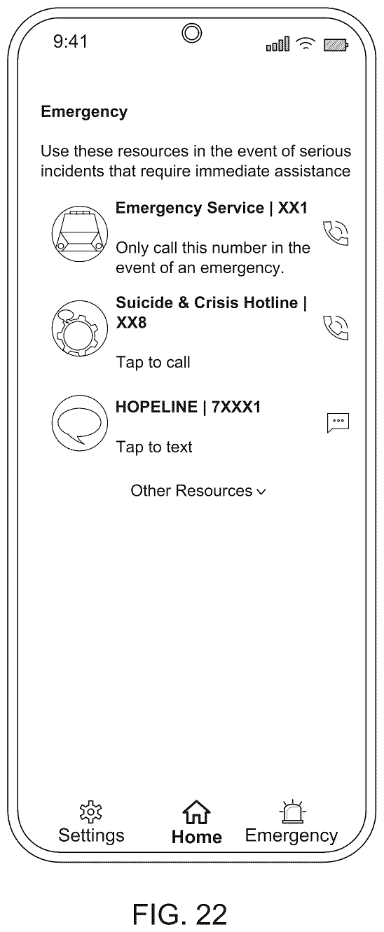

FIG. 22 is a diagram illustrating an exemplary user interface for accessing emergency resources according to embodiments of the subject matter described herein. FIG. 22 showcases the Emergency Resources section available to clinicians, demonstrating the system's commitment to patient safety. This section provides quick access to essential contact information and protocols for handling emergencies, empowering clinicians to respond effectively in critical situations.

### Administrator-Side

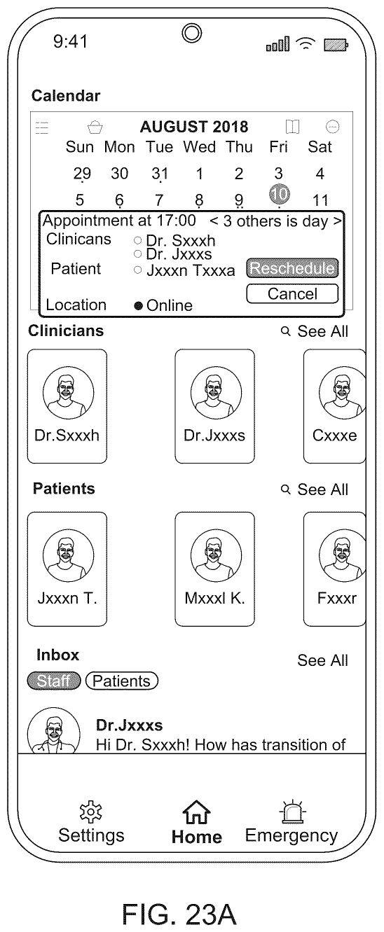

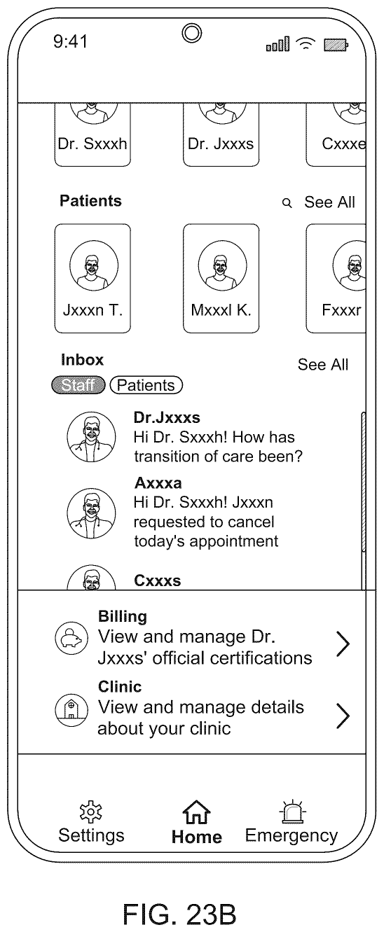

FIGS. 23A-26 highlight administrator functions, such as editing clinician profiles, scheduling events, and managing hospital records. Interfaces are organized by task type, with dropdown menus or input fields for detailed edits.

FIGS. 23A and 23B are diagrams illustrating exemplary home screen user interfaces for managing administrator functions according to embodiments of the subject matter described herein. Specifically, FIGS. 23A and 23B depict the administrator-side interface including the centralized hub designed for managing various administrative functions within the system. These figures illustrate the dashboard view, providing administrators with an overview of system usage, user statistics, and potential alerts that require attention. This centralized platform streamlines administrative tasks, ensuring smooth operation and efficient management of the system.

### Administrator-Side: Clinician View

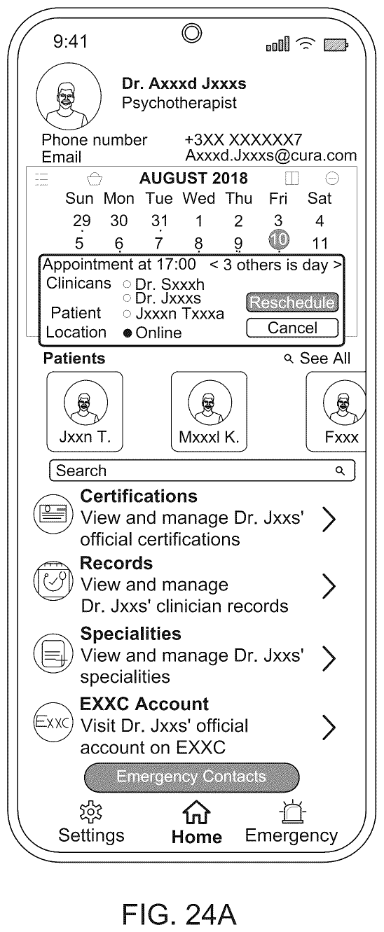

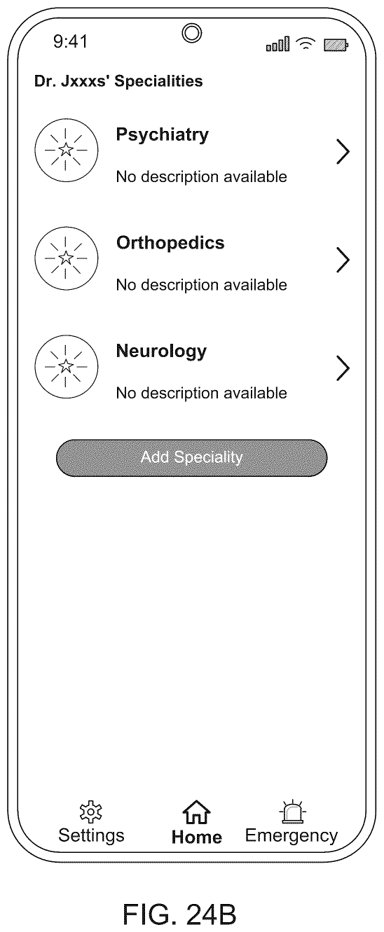

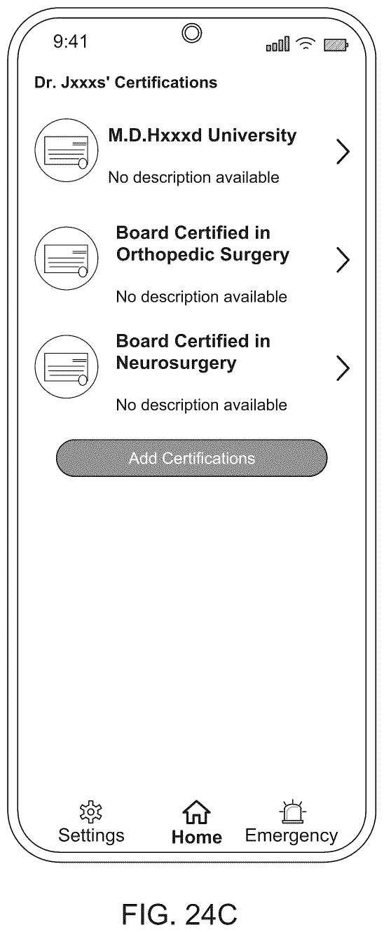

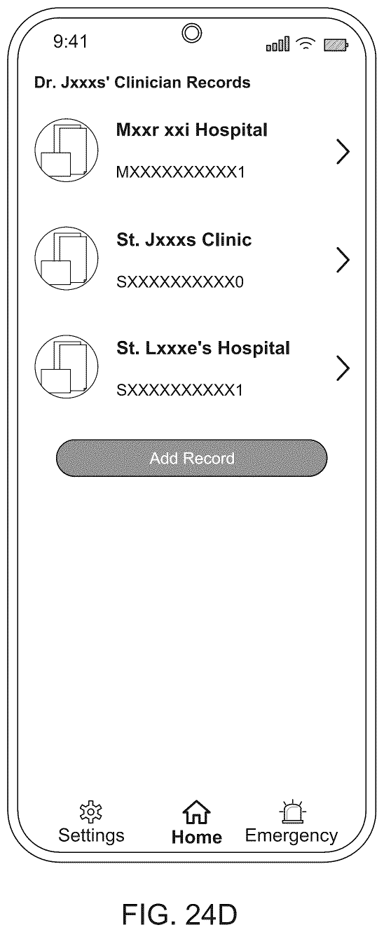

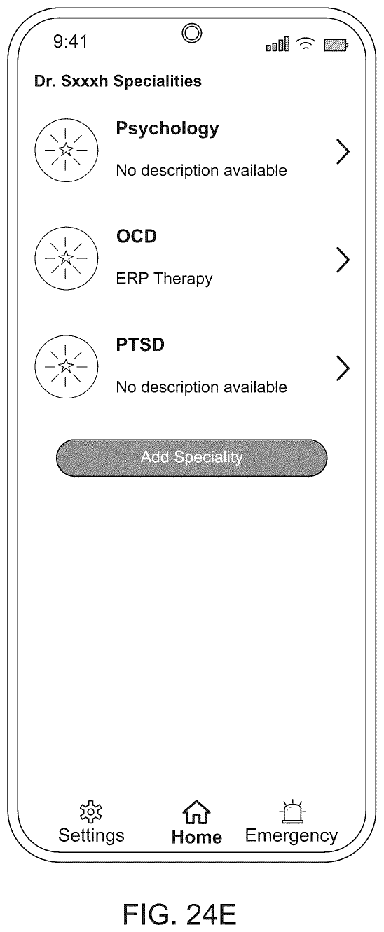

FIGS. 24A, 24B, 24C, 24D, and 24E are diagrams illustrating exemplary user interfaces for viewing a clinician's profile, adding appointments and events to a schedule, updating records, and viewing a clinician's specialties according to embodiments of the subject matter described herein. FIGS. 24A to 24E illustrate the clinician management features accessible to administrators, demonstrating the system's role in supporting efficient staff management and resource allocation. Here, the certifications and specialties for two different doctors are shown. These can be added to or edited by an administrator. It is appreciated that some functions, such as these, may only be available to administrators and not to other types of users (e.g., patients) and may be determined by an access control list or other user profile.

FIG. 24A shows a clinician's profile, providing administrators with access to their contact information, credentials, and areas of specialization.

FIG. 24B depicts the interface for managing a clinician's schedule, allowing administrators to view, add, or edit appointments and events.

FIG. 24C demonstrates the functionality for updating a clinician's records, allowing administrators to maintain accurate and up-to-date information regarding certifications, licenses, and other relevant credentials.

FIG. 24D provides an overview of a clinician's specialties, allowing administrators to assign patients based on the clinician's expertise and experience.

FIG. 24E provides an alternative view of a clinician's specialties, demonstrating the system's flexibility in presenting information. These features collectively empower administrators to effectively manage clinician profiles, ensuring that patients are matched with appropriately qualified professionals and that the clinic operates smoothly.

### Administrator-Side: Hospital Information

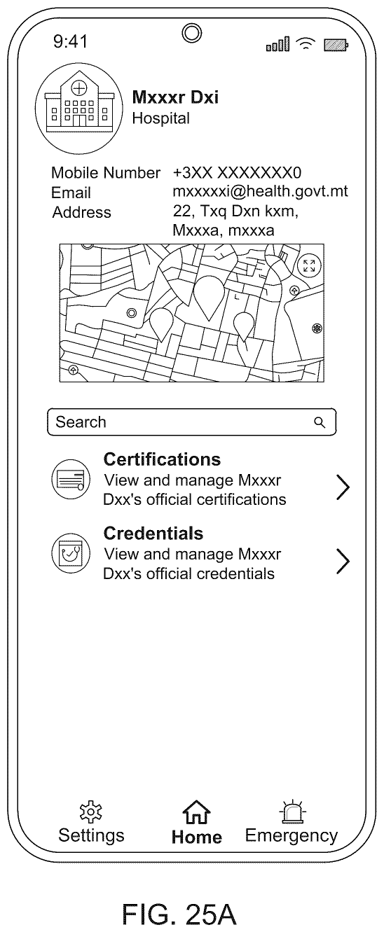

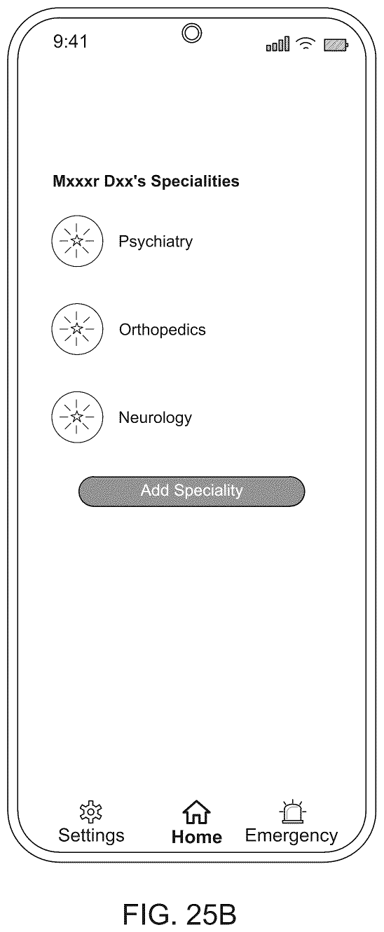

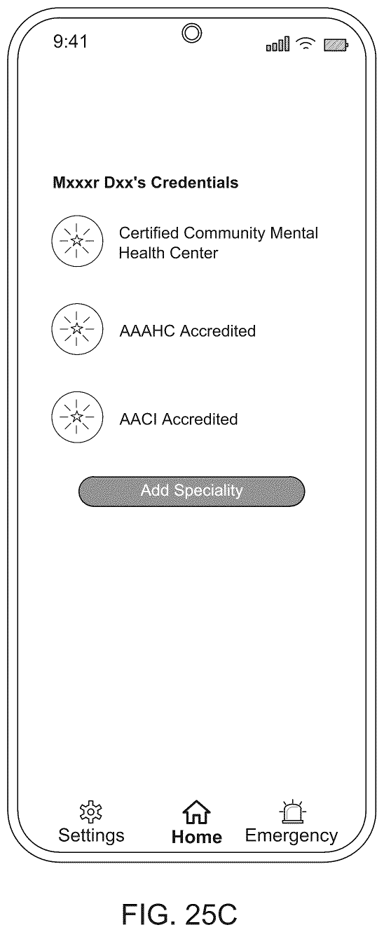

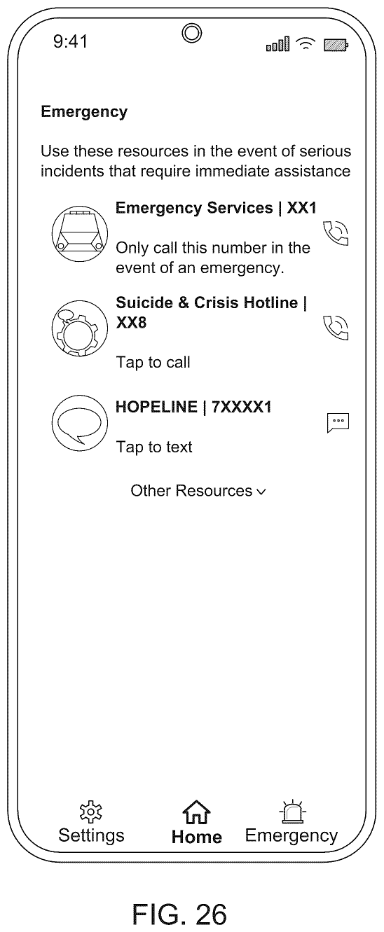

FIGS. 25A to 26 focus on the administrator's role in managing system-wide information, ensuring accuracy and consistency of data crucial for the platform's functionality. FIGS. 25A, 25B, and 25C are diagrams illustrating exemplary user interfaces for viewing, adding, and editing hospital information according to embodiments of the subject matter described herein.

FIG. 25A provides an overview of existing hospital entries, allowing administrators to quickly review and access relevant details.

FIG. 25B illustrates the process of adding new hospital information, ensuring that the system database remains current and comprehensive.

FIG. 25C demonstrates the functionality for editing existing hospital information, allowing administrators to update details as needed, maintaining accuracy and reflecting any changes in hospital operations or services. This meticulous management of hospital information ensures that clinicians and patients have access to reliable and up-to-date information regarding appointment locations, services offered, and other crucial details that might influence their healthcare decisions.

### Administrator-Side: Emergency Resources

FIG. 26 is a diagram illustrating an exemplary user interface for accessing emergency resources according to embodiments of the subject matter described herein. FIG. 26 highlights the Emergency Resources section specifically designed for administrators. This section provides administrators with quick access to contact information and protocols for handling system-wide emergencies, such as data breaches, server outages, or other critical events. This dedicated resource ensures that administrators can respond swiftly and effectively to unexpected situations, minimizing potential disruptions and ensuring the platform's security and reliability.

In other possible embodiments, “digital twins” may be used in which the user's collected data is analyzed using artificial intelligence and machine learning algorithms in order to create a model which accurately represents the user's degree of obsessive-compulsive disorder, mental health and other elements tracked through the app.

In other possible embodiments, artificial intelligence may be used to perform translation and automatic alternative text generation, thereby increasing the accessibility and globalization of the subject matter described herein.

As will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium (including, but not limited to, non-transitory computer readable storage media). A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

Computer program code for carrying out operations for aspects of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter situation scenario, the remote computer may be connected to the user's computer through any type of network, including a local area network (LAN) or a wide area network (WAN), or the connection may be made to an external computer (for example, through the Internet using an Internet Service Provider).

Aspects of the present invention are described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

The flowchart and block diagrams in the FIGS. illustrate the architecture, functionality, and operation of possible implementations of systems, methods and computer program products according to various embodiments of the present invention. In this regard, each block in the flowchart or block diagrams may represent a module, segment, or portion of code, which comprises one or more executable instructions for implementing the specified logical function(s). It should also be noted, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The terminology used herein is for the purpose of describing particular embodiments only and is not intended to be limiting of the invention. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A computer-implemented method for managing exposure and response prevention (ERP) treatment healthcare workflows of a patient, the computer-implemented method comprising instructions stored on a non-transitory computer-readable storage medium and executed on a computing device provided with a hardware processor and a memory, the method comprising:
receiving, by the computing device, healthcare data from a plurality of sources, wherein the healthcare data includes at least: symptoms reported by the patient, treatment history, and clinician-provided instructions;
processing the healthcare data to generate a treatment plan, wherein the treatment plan includes exposure assignments tailored to the patient, a sequence of ritual resistance tasks, and anxiety level monitoring parameters;
presenting, via a graphical user interface (GUI) rendered on the computing device, at least one interactive display to the patient, wherein the GUI is configured to:
display progress metrics related to completion of the exposure assignments and ritual resistance tasks;
highlight upcoming tasks aligned with predicted anxiety windows; and
provide clinician feedback generated in part by analyzing patient interaction data using a trained machine learning model;
receiving, by the computing device, user input from the patient or a clinician indicating completion, modification, or deferral of specific tasks within the treatment plan; and
updating, by the hardware processor according to predefined ERP treatment guidelines, at least one subsequent exposure assignment or ritual resistance task in response to the received user input.

2. The method of claim 1, wherein processing the healthcare data to generate a treatment plan includes:
applying, by the hardware processor, the trained machine learning model configured to analyze the healthcare data to:
detect patterns indicative of anxiety triggers of the patient relevant to exposure and response prevention therapy;
predict changes in an anxiety level of the patient over a defined timeframe; and
determine personalized modifications to an ERP treatment plan based on probabilistic inferences drawn from the detected patterns; and
generating, based on an output of the trained machine learning model, a treatment plan that is specific to ERP therapy, wherein the treatment plan comprises:
exposure assignments tailored to the patient's identified anxiety triggers;
a sequence of ritual resistance tasks configured to gradually reduce compulsive responses; and
anxiety level monitoring parameters that adapt in real time according to predictive outputs of the machine learning model.

3. The method of claim 1, wherein the trained machine learning model is further invoked to:
incorporate real-time input as additional data points to refine predicted anxiety trajectories; and
dynamically adjust a schedule and an intensity of the treatment plan for future exposure tasks.

4. The method of claim 1, wherein processing the healthcare data includes correlating patient-reported anxiety levels with historical treatment data to determine an optimal exposure intensity.

5. The method of claim 1, wherein the GUI includes separate views optimized for patients, clinicians, and administrators, each tailored to display role-specific information.

6. The method of claim 1, wherein the received input includes a user-selected indication of ritual resistance or submission during a specific exposure assignment.

7. The method of claim 1, wherein the updated treatment plan includes new exposure tasks derived from a hierarchy of fears established during an initial patient assessment.

8. The method of claim 1, wherein the GUI includes a visual progress tracker that graphically represents the patient's completion of assigned tasks over time.

9. The method of claim 1, further comprising providing automated alerts to a clinician when a patient fails to complete an assigned task within a designated timeframe.

10. The method of claim 1, wherein the healthcare data is supplemented by external sources, including medication records, clinician notes, and appointment schedules.

11. A system for managing exposure and response prevention (ERP) treatment healthcare workflows of a patient, comprising:
a computing device communicatively coupled to a database, the computing device including
a hardware processor and a memory storing instructions, wherein execution of the instructions by the hardware processor causes the computing device to:
receive, from a plurality of sources, healthcare data that includes patient-provided symptom logs, treatment history, and clinician-generated records relating to ERP;
process the healthcare data to generate a treatment plan, wherein the treatment plan includes a sequence of exposure assignments, ritual resistance tracking, and anxiety level monitoring parameters;
present, via a graphical user interface (GUI) executable on the computing device, the generated treatment plan to at least one of the patient, a clinician, or an administrator, wherein the GUI is further configured to:
display progress metrics related to the patient's completion of exposure assignments and ritual resistance tasks;
provide a timeline or sequence of upcoming tasks based on predicted anxiety windows; and
incorporate feedback from the clinician or patient regarding the assigned tasks;
receive user input, including completion data or modification requests relating to at least one exposure assignment or ritual resistance task; and
update the treatment plan dynamically based on predefined rules and the received user input.

12. The system of claim 11, wherein processing the healthcare data to generate the treatment plan includes:
applying, by the hardware processor, a trained machine learning model configured to analyze the healthcare data to:
detect patterns indicative of anxiety triggers of the patient's relevant to exposure and response prevention therapy;
predict changes in an anxiety level of the patient over a defined timeframe; and
determine personalized modifications to an ERP treatment plan based on probabilistic inferences drawn from the detected patterns; and
generating, based on an output of the trained machine learning model, a treatment plan that is specific to ERP therapy, wherein the treatment plan comprises:
exposure assignments tailored to the patient's identified anxiety triggers;
a sequence of ritual resistance tasks configured to gradually reduce compulsive responses; and
anxiety level monitoring parameters that adapt in real time according to predictive outputs of the machine learning model.

13. The system of claim 12, wherein the trained machine learning model is further invoked to:
incorporate real-time input as additional data points to refine predicted anxiety trajectories; and
dynamically adjust the treatment plan's scheduling and intensity for future exposure tasks.

14. The system of claim 11, wherein the database includes structured data fields for patient anxiety metrics, treatment goals, and task progress tracking.

15. The system of claim 11, wherein the computing device assigns incremental exposure tasks to the patient based on hierarchical fear data established during initial setup.

16. The system of claim 11, wherein the GUI is dynamically configured to provide a progress tracker, exposure task status, and upcoming assignments for patients.

17. The system of claim 11, wherein the computing device stores ritual resistance data as timestamped records, enabling clinicians to analyze trends over time.

18. The system of claim 11, wherein the computing device generates notifications for clinicians when patient task completion rates fall below a predefined threshold.

19. The system of claim 11, wherein the treatment plan updates include algorithmically generated recommendations for new exposure assignments based on patient progress and clinician-defined parameters.

20. The system of claim 11, wherein the GUI includes an administrator dashboard for managing user roles, clinician assignments, and audit trails of treatment modifications.
